(19) United States
(12) Patent Application Publication
(10) Pub. No.: US 2006/0057694 A1
(43) Pub. Date: Mar. 16, 2006

(54) Rat receptor tyrosine knase, kdr

(76) Inventors: Kenneth A Thomas JR., Chatham, NJ (US); Bo-Sheng Pan, Lansdale, PA (US); Georgia B McGaughey, Harleysville, PA (US)

Correspondence Address: MERCK AND CO., INC, P O BOX 2000, RAHWAY, NJ, 07065-0907, US

(21) Appl. No.: 10/541,814

(22) PCT Filed: Jan. 23, 2004
(86) PCT No.: PCT/US04/01928

§ 371 Date: Jul. 6, 2005

Related U.S. Application Data
(60) Provisional application No. 60/443,335, filed on Jan. 29, 2003.

(51) Int. Cl. C12N 9/12; C07H 21/04; C12P 21/06; C12N 5/06

(52) U.S. Cl. 435/183; 435/194; 435/353; 435/320.1; 536/23.2; 435/69.1

(57) ABSTRACT

An isolated nucleic acid molecule encoding an optimized rat receptor type tyrosine kinase, KDR, is disclosed. The isolation of this KDR cDNA sequence results in disclosure of purified forms of rat KDR protein, recombinant vectors and recombinant hosts which express rat KDR.

### CROSS-REFERENCE TO RELATED APPLICATIONS

The present application claims the benefit of U.S. Provisional Application No. 60/443,335, filed Jan. 29, 2003, hereby incorporated by reference herein.

### STATEMENT REGARDING FEDERALLY-SPONSORED R&D

Not applicable

### REFERENCE TO MICROFICHE APPENDIX

Not applicable

### FIELD OF THE INVENTION

The present invention relates to an isolated nucleic acid molecule (polynucleotide) which encodes a rat receptor tyrosine kinase, KDR. This receptor is expressed on rat endothelial cells and is activated by VEGF to mediate a mitogenic signal. The present invention also includes: recombinant vectors and recombinant hosts which contain a DNA fragment encoding rat KDR; DNA fragments encoding the intracellular portion of KDR; DNA fragments encoding the extracellular portion of KDR with or without a membrane anchor; substantially purified forms of associated rat KDR; and rat mutant forms of KDR.

### BACKGROUND OF THE INVENTION

In vascular endothelial cells, mitogens promote embryonic vascular development, growth, repair and angiogenesis. One class of mitogens selective for vascular endothelial cells include vascular endothelial growth factor (referred to as VEGF or VEGF-A) and the homologues placenta growth factor (PlGF), VEGF-B and VEGF-C. VEGF and its homologues exert their endothelial specific mitogenic effect by binding to vascular endothelial cell plasma membrane-spanning tyrosine kinase receptors which then activate an intracellular mitogenic signal. The KDR receptor family is the major tyrosine kinase receptor which transduces the mitogenic signal initiated by VEGF. Inhibiting KDR significantly diminishes the level of mitogenic VEGF activity.

Vascular growth in the retina leads to visual degeneration culminating in blindness. VEGF accounts for most of the angiogenic activity produced in or near the retina in diabetic retinopathy.

Expression of VEGF is also significantly increased in hypoxic regions of animal and human tumors adjacent to areas of necrosis. Monoclonal and polyclonal anti-VEGF antibodies inhibit the growth of tumors in nude mice. Embryonic stem cells, which normally grow as solid tumors in nude mice, do not produce detectable tumors if both VEGF alleles are knocked out. Taken together, these data indicate the role of VEGF in the growth of solid tumors.

As the KDR receptor family of tyrosine kinase receptors is implicated in pathological neoangiogenesis, inhibitors of these receptors are useful in the treatment of diseases in which neoangiogenesis is part of the overall pathology, e.g., diabetic retinal vascularization, various forms of cancer as well as forms of inflammation such as rheumatoid arthritis, psoriasis, contact dermatitis and hypersensitivity reaction.

U.S. Pat. No. 6,204,011 discloses an optimized human KDR nucleotide and amino acid sequence.

Wen et al. (1998, J. Biol. Chem. 273: 2090-2097) disclose a full-length cDNA encoding a form of rat KDR. However, the Wen et al. disclosures do not identify a novel, optimal nucleic acid fragment encoding the rat form of the receptor type tyrosine kinase gene, KDR. It will be advantageous to identify and isolate a rat cDNA sequence encoding an optimized form of rat KDR. A nucleic acid molecule expressing the rat KDR protein will be useful in screening for compounds acting as modulators of the protein kinase domain of this receptor in rats. Such a compound or compounds can be used in modulating the mitogenic signal of VEGF and VEGF-related proteins on vascular endothelial cells. Inhibitors of rat KDR will be useful to treat human diseases including cancer, ischemic ocular diseases such as proliferative diabetic retinopathy, and inflammation. Either all or a portion of the KDR protein is also useful to screen for VEGF antagonists. Furthermore, the KDR protein can be used for x-ray structure analysis in the presence or absence of ligand and/or inhibitors. The present invention addresses and meets these needs by disclosing an isolated nucleic acid fragment which expresses a form of rat KDR which is experimentally shown to have a higher activity and functionality than the previously disclosed KDR.

### SUMMARY OF THE INVENTION

The present invention relates to an isolated nucleic acid molecule (polynucleotide) which encodes an optimized rat receptor type tyrosine kinase, KDR, a receptor tyrosine kinase expressed on rat endothelial cells.

The present invention further relates to an isolated nucleic acid molecule (polynucleotide) which encodes a rat receptor type tyrosine kinase, KDR, this nucleic acid molecule comprising a nucleotide sequence encoding a rat KDR retaining Asp at position 1083, and alternatively retaining Asp at position 1083 in combination with Ala at position 1061, Val at position 1077, and/or Glu at position 1110.

The present invention also relates to an isolated nucleic acid molecule (polynucleotide) which encodes a rat receptor type tyrosine kinase, KDR, this nucleic acid molecule comprising a nucleotide sequence encoding the amino acid sequence as disclosed in FIG. 2 and as set forth in SEQ ID NO:2.

The present invention also relates to an isolated nucleic acid molecule (polynucleotide) comprising the DNA molecule as disclosed in FIGS. 1A-D and as set forth in SEQ ID NO:1, which encodes a rat receptor type tyrosine kinase, KDR, as disclosed in FIG. 2 and as set forth in SEQ ID NO:2.

The present invention relates to an isolated nucleic acid molecule (polynucleotide) which encodes a rat receptor type tyrosine kinase, KDR, this nucleic acid molecule consisting of a nucleotide sequence encoding the amino acid sequence as disclosed in FIG. 2 and as set forth in SEQ ID NO:2.

The present invention also relates to an isolated nucleic acid molecule (polynucleotide) consisting of the DNA molecule as disclosed in FIGS. 1A-D and as set forth in SEQ ID NO:1, which encodes a rat receptor type tyrosine kinase, KDR, as disclosed in FIG. 2 and as set forth in SEQ ID NO:2.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to biologically active fragments or mutants of SEQ ID NO:1 which encode mRNA expressing an optimized rat receptor type tyrosine kinase gene, KDR. Any such biologically active fragment and/or mutant will encode either a protein or protein fragment comprising at least an intracellular or extracellular domain similar to that of the rat KDR protein as set forth in SEQ ID NO:2. Any such polynucleotide includes but is not necessarily limited to nucleotide substitutions, deletions, additions, amino-terminal truncations and carboxyl-terminal truncations such that these mutations encode mRNA which express a protein or protein fragment of diagnostic, therapeutic or prophylactic use and would be useful for screening for agonists and/or antagonists for KDR function.

The present invention also relates to isolated nucleic acid molecules which encode rat KDR protein fragments comprising a portion of the intracellular KDR domain, said protein fragments retaining Asp at position 1083, and alternatively retaining Asp at position 1083 in combination with Ala at position 1061, Val at position 1077, and/or Glu at position 1110. The protein fragments are useful in assays to identify compounds which modulate wild-type rat KDR activity. A preferred aspect of this portion of the invention includes, but is not limited to, a nucleic acid construction which encodes the intracellular portion of rat KDR, from about amino acid 765-785 to about amino acid 1156-1343.

The present invention also relates to isolated nucleic acid molecules which encode rat KDR protein fragments comprising a portion of the extracellular KDR domain, and may or may not include nucleotide sequences which also encode the transmembrane domain of rat KDR. Said protein fragments will retain Asn at position 519, Gln at position 560, Val at position 563, Ala at position 753, Val at position 781, and/or Leu at position 782. These KDR extracellular and/or KDR extracellular-transmembrane domain protein fragments will be useful in screening for compounds which inhibit VEGF binding.

The present invention also relates to isolated nucleic acid molecules which are fusion constructions expressing fusion proteins useful in assays to identify compounds which modulate wild-type rat KDR activity. A preferred aspect of this portion of the invention includes, but is not limited to, glutathione S-transferase (GST)-KDR fusion constructs. These fusion constructs include, but are not limited to, either the intracellular tyrosine kinase domain of rat KDR as an in-frame fusion at the carboxy terminus of the GST gene or the extracellular ligand binding domain fused to an immunoglobin gene by methods known to one of ordinary skill in the art. Soluble recombinant GST-kinase domain fusion proteins may be expressed in various expression systems, including Spodoptera frugiperda (Sf21) insect cells (Invitrogen) using a baculovirus expression vector (pAcG2T, Pharmingen).

The present invention also relates to recombinant vectors and recombinant hosts, both prokaryotic and eukaryotic, which contain the substantially purified nucleic acid molecules disclosed throughout this specification.

The present invention relates to a purified form of an optimized rat receptor type tyrosine kinase protein, KDR, a receptor tyrosine kinase expressed on rat endothelial cells.

The present invention further relates to a purified form of a rat receptor type tyrosine kinase protein, KDR, comprising an amino acid sequence retaining Asp at position 1083, and alternatively retaining Asp at position 1083 in combination with Ala at position 1061, Val at position 1077, and/or Glu at position 1110.

The present invention also relates to a purified form of a rat receptor type tyrosine kinase protein, KDR, comprising the amino acid sequence as disclosed in FIG. 2 and as set forth in SEQ ID NO:2.

The present invention also relates to a purified form of a rat receptor type tyrosine kinase protein, KDR, consisting of the amino acid sequence as disclosed in FIG. 2 and as set forth in SEQ ID NO:2.

The present invention also relates to biologically active fragments and/or mutants of the KDR protein as initially set forth as SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists for KDR function.

The present invention further relates to subcellular membrane fractions of the recombinant host cells (both prokaryotic and eukaryotic as well as both stably and transiently transformed cells) comprising the nucleic acids of the present invention. These subcellular membrane fractions will comprise either wild-type or rat mutant forms of KDR at levels substantially above wild-type levels and hence will be useful in various assays described throughout this specification.

The present invention also relates to polyclonal and monoclonal antibodies raised in response to either the rat form of KDR disclosed herein, or a biologically active fragment thereof.

Therefore, the present invention relates to methods of expressing the receptor type tyrosine kinase gene, KDR, and biological equivalents disclosed herein, assays employing these receptor type tyrosine kinase genes, and cells expressing these receptor type tyrosine kinase genes. The present invention also relates to compounds identified through the use of these receptor type tyrosine kinase genes and expressed rat KDR protein, including one or more modulators of the rat KDR-dependent kinase either through direct contact with the kinase domain of rat KDR or a compound which prevents binding of VEGF to rat KDR, or appropriate dimerization of the KDR receptor antagonizing transduction of the normal intracellular signals associated with VEGF-induced angiogenesis.

As used herein, “VEGF” or “VEFG-A” refers to vascular endothelial growth factor.

As used herein, “KDR” refers to kinase insert domain-containing receptor.

As used herein, the term “mammalian host” refers to any mammal, including a human being.

### BRIEF DESCRIPTION OF THE DRAWINGS

FIGS. 1A-D shows the nucleotide sequence which encodes an optimized rat KDR, as set forth in SEQ ID NO:1.

FIG. 2 shows the amino acid sequence of an optimized rat KDR, as also set forth in SEQ ID NO:2. Underlined amino acid residues represent differences in comparison to a previously disclosed form of rat KDR.

FIG. 3A and FIG. 3B show an alignment comparing the rat KDR amino acid sequence published in the National Center for Biotechnology Information (NCBI) protein database (accession no. O08775; SEQ ID NO:15) with the optimized rat KDR amino acid sequence of the present invention. The amino acid differences of the optimized rat KDR of the present invention when compared to the published rat KDR sequence are underlined.

FIG. 4 shows the crystal structure of human KDR with substrate, specifically denoting the location of four amino acids, Ala (A) at position 1065, Val (V) at position 1081, Asp (D) at position 1087 and Glu (E) at position 1114. These four residues are conserved between human KDR and the optimized rat KDR of the present invention.

FIG. 5 shows a magnified view a region of the crystal structure of human KDR encompassing the Asp residue at position 1087 of the sequence. Asp 1087 is hydrogen bonded to two backbone amide protons in the catalytic loop, His-1026 and Arg-1027.

FIG. 6 shows the effect of a Gly residue at position 1083 (G1083) within the kinase domain of rat KDR on its ability to autophosphorylate. RK7, a fragment encoding the intracellular kinase domain of optimized rat KDR, was altered to contain a Gly residue at position 1083. Purified GST-RK7 (G1083) was unable to autophosphorylate in the presence of 1 mM ATP; however, purified GST-RK7 exhibited rapid autophosphorylation.

FIG. 7 shows the effect of a Gly residue at position 1083 (G1083) within the kinase domain of rat KDR on its ability to tyrosine-phosphorylate a synthetic biotinylated peptide substrate. Purified GST-RK7 (G1083) showed no detectable tyrosine kinase activity (open circles), while GST-RK7 tyrosine-phosphorylated the peptide substrate (closed squares).

FIG. 8A shows the nucleotide sequence which encodes a GST-tagged rat KDR fusion protein, labeled GST-RK7, as also set forth in SEQ ID NO:17. The nucleotide sequence encoding RK7, a fragment encoding the intracellular kinase domain of optimized rat KDR, is located 3′ of the nucleotide sequence encoding GST. Located within the GST coding region is a 6×-histidine tag.

FIG. 8B shows the amino acid sequence of GST-RK7, as also set forth in SEQ ID NO:18.

### DETAILED DESCRIPTION OF THE INVENTION

The present invention relates to isolated nucleic acid and protein forms which represent an optimized rat KDR. This specification discloses a DNA molecule encoding an optimized rat KDR, a receptor tyrosine kinase expressed on rat endothelial cells. The receptor is activated by vascular endothelial growth factor (VEGF) and mediates a mitogenic signal. This activation and subsequent mitogenesis leads to an angiogenic response in vivo.

The present invention further relates to an isolated nucleic acid molecule (polynucleotide) which encodes a rat receptor type tyrosine kinase, KDR, this nucleic acid molecule comprising a nucleotide sequence encoding a rat KDR retaining Asp at position 1083, and alternatively retaining Asp at position 1083 in combination with Ala at position 1061, Val at position 1077, and/or Glu at position 1110. The nucleic acid molecule disclosed in the specification as SEQ ID NO:1 encodes a rat KDR protein (SEQ ID NO:2) which results in ten amino acid differences from the published sequence (Wen et al., J. Biol. Chem. 273:2090-2097; NCBI GenBank accession no. O08775). Four of these changes are located within the intracellular kinase domain of the rat KDR protein, specifically at positions 1061 (Pro to Ala), 1077 (Ile to Val), 1083 (Gly to Asp) and 1110 (Lys to Glu). These four amino acids are conserved throughout most of the tyrosine kinase family. Of the four intracellular amino acid differences, the Asp (D) residue at position 1083 affects the activity of the receptor. The homologous residue in human KDR (D1087) has been shown to be structurally close to the catalytic loop of the protein which mediates phosphotransfer. A Gly (G) located at the corresponding position in the rat KDR sequence, position 1083, results in a non-functional kinase. The other residue changes located within the intracellular kinase domain may also cause activity differences. The residue in human KDR corresponding to the Ala at position 1061 of the optimized rat sequence of the present invention is located within the activation loop. A change from Ala to Pro at this position is likely to reduce the flexibility of the activation loop which is required for kinase activity. The remaining amino acid differences are located within the extracellular or transmembrane domain of the rat KDR protein. Specifically, four of the amino acids changes are located within the extracellular domain at positions 519 (Tyr to Asn), 560 (Arg to Gln), 563 (Met to Val), and 753 (Val to Ala). Since the mitogenic activity of KDR is initiated by binding of VEGF to the extracellular domain of the receptor, these specific amino acid differences may alter the binding of VEGF, its homologues, and any KDR agonists and/or antagonists that modulate KDR activity. The four amino acid changes located within the extracellular domain are present in the human and mouse KDR sequences, suggesting that these residues may be structurally or functionally important. The two remaining amino acid differences are located within the short transmembrane domain of KDR, specifically at position 781 (Leu to Val) and position 782 (Val to Leu).

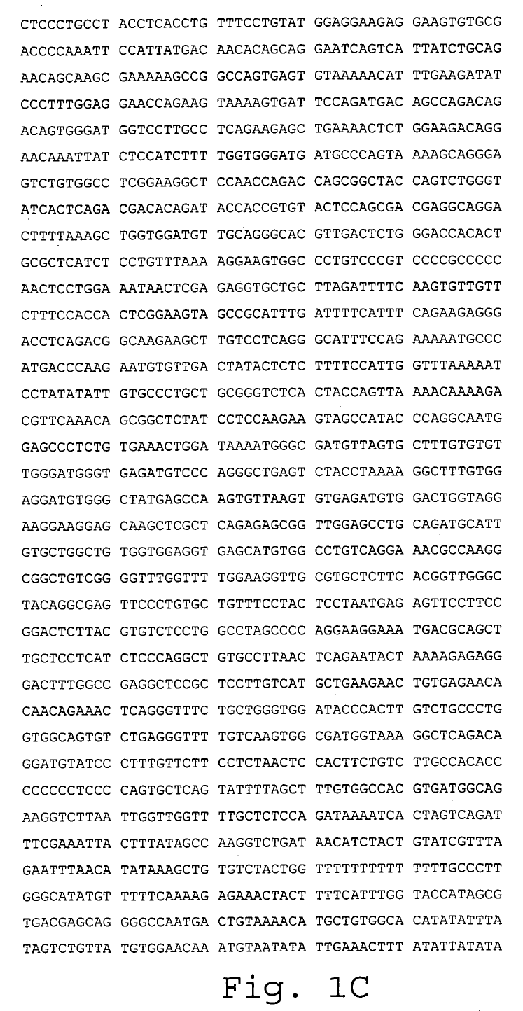

The present invention also relates to an isolated nucleic acid molecule (polynucleotide) which encodes a rat receptor type tyrosine kinase, KDR, this nucleic acid molecule comprising or consisting of a nucleotide sequence encoding the amino acid sequence as disclosed in FIG. 2 and as set forth in SEQ ID NO:2. The amino acid sequence set forth in SEQ ID NO:2 encompasses the ten amino acid differences that exist between the optimized rat KDR of the present invention and the published rat KDR sequence. Therefore, the present invention includes codon redundancy which may result in differing DNA molecules expressing an identical protein.

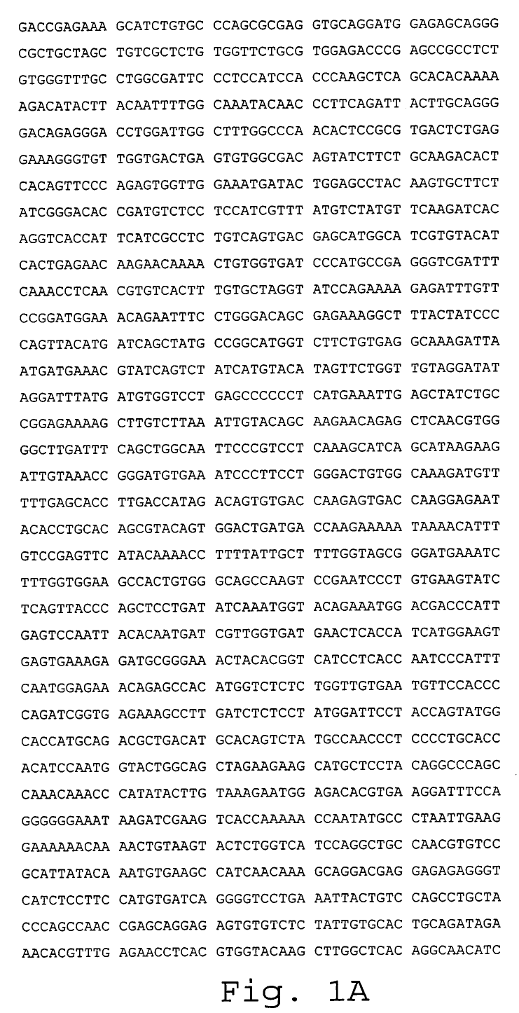

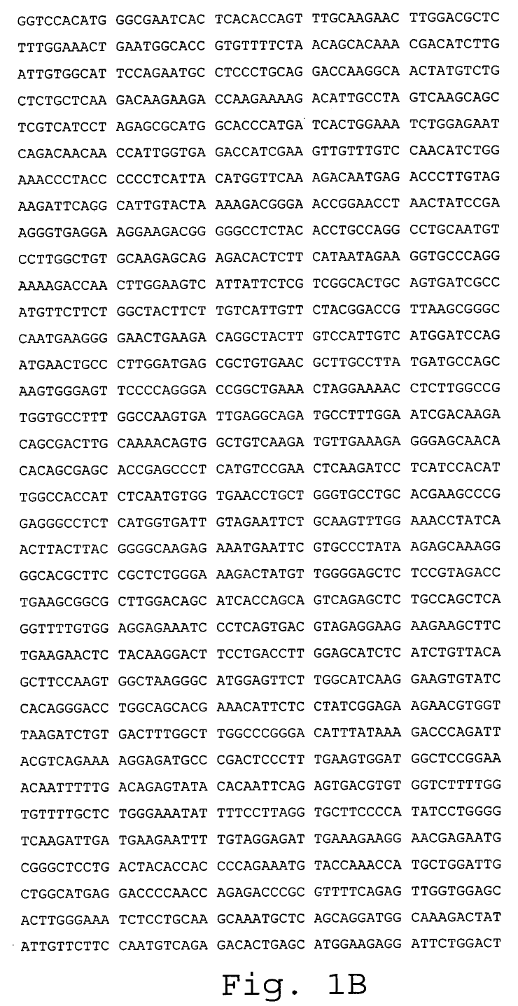

The present invention further relates to an isolated nucleic acid molecule (polynucleotide) comprising or consisting of the DNA molecule as disclosed in FIGS. 1A-D and as set forth in SEQ ID NO:1, which encodes the rat KDR as disclosed in FIG. 2 and as set forth in SEQ ID NO:2.

The present invention also relates to either biologically active fragments or mutants of SEQ ID NO:1 which encode mRNA expressing a novel rat receptor type tyrosine kinase gene, KDR. Any such biologically active fragment and/or mutant will encode a protein or protein fragment comprising at least an intracellular or extracellular domain similar to that of the rat KDR protein as set forth in SEQ ID NO:2. Any such protein fragment may be a fusion protein, such as a GST-tagged KDR fusion protein, or may be solely comprised of the KDR intracellular domain, with increasing deletions in from the COOH-terminal region. It is especially preferable that the following amino acids be retained if the fragment encompasses the respective protein domain: Asn at position 519, Gln at position 560, Val at position 563, Ala at position 753, Val at position 781, Leu at position 782, Asp at position 1083, Ala at position 1061, Val at position 1077 and/or Glu at position 1110. Therefore, any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and is useful for the identification of modulators of KDR receptor activity.

Therefore, the present invention relates to isolated nucleic acid molecules which encode rat KDR protein fragments comprising a portion of the intracellular kinase domain. Any such nucleic acid will encode a KDR protein fragment which mimics KDR wild-type kinase activity. The protein fragments are useful in assays to identify compounds which modulate wild-type rat KDR activity. A preferred aspect of this portion of the invention includes, but is not limited to, a nucleic acid construction which encodes the intracellular portion of optimized rat KDR from about amino acid 765-785 to about amino acid 1156-1343, retaining Asp at position 1083, and alternatively retaining Asp at position 1083 in combination with Ala at position 1061, Val at position 1077, and/or Glu at position 1110. These expressed soluble protein fragments may or may not contain a portion of the amino-terminal region of rat KDR or of a heterologous sequence. These nucleic acids may be expressed in any of a number of expression systems available to the artisan.

The present invention also relates to isolated nucleic acid molecules which encode rat KDR protein fragments comprising a portion of the extracellular domain. These isolated nucleic acid may or may not include nucleotide sequences which also encode the transmembrane domain of rat KDR located from amino acid residue 761 to amino acid residue 782. Said protein fragments will retain Asn at position 519, Gln at position 560, Val at position 563, Ala at position 753, Val at position 781, and/or Leu at position 782. These KDR extracellular and/or KDR extracellular-transmembrane domain protein fragments will be useful in screening for compounds which inhibit VEGF binding. Expression of either a soluble version of KDR (extracellular) or membrane bound form (extracellular-transmembrane) will inhibit VEGF/KDR mediated angiogenesis.

The present invention also relates to isolated nucleic acid molecules which are fusion constructions useful in assays to identify compounds which modulate wild-type rat KDR activity. Such assays can be used to evaluate the safety and efficacy of specific inhibitors of KDR in rats. These inhibitors will be useful to treat human diseases including cancer, ischemic ocular diseases such as proliferative rentinopathy, and inflammation. A preferred aspect of this portion of the invention includes, but is not limited to, GST-KDR fusion constructs. These fusion constructs comprise the intracellular tyrosine kinase domain of rat KDR as an in-frame fusion at the carboxy terminus of the GST gene. An exemplified GST-tagged rat KDR fusion protein, GST-RK7, is described in Example 5 and set forth in SEQ ID NO:18. RK7 represents a fragment of the optimized rat KDR encoding the intracellular kinase domain. The nucleotide sequence encoding RK7 is located 3′ of the nucleotide sequence encoding GST, as set forth in SEQ ID NO:17. Located within the GST coding region is a 6×-histidine tag. Soluble recombinant GST-kinase domain fusion proteins may be expressed in various expression systems, including Spodoptera frugiperda (Sf21) insect cells (Invitrogen) using a baculovirus expression vector (pAcG2T, Pharmingen).

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The degeneracy of the genetic code is such that, for all but two amino acids, more than a single codon encodes a particular amino acid. This allows for the construction of synthetic DNA that encodes the optimized rat KDR protein where the nucleotide sequence of the synthetic DNA differs significantly from the nucleotide sequence of SEQ ID NO:1 but still encodes the same optimized rat KDR protein of SEQ ID NO:2. Such synthetic DNAs are intended to be within the scope of the present invention. If it is desired to express such synthetic DNAs in a particular host cell or organism, the codon usage of such synthetic DNAs can be adjusted to reflect the codon usage of that particular host, thus leading to higher levels of expression of the rat KDR protein in the host. In other words, this redundancy in the various codons which code for specific amino acids is within the scope of the present invention. Therefore, the present invention discloses codon redundancy which may result in differing DNA molecules expressing an identical protein.

It is known that DNA sequences coding for a peptide may be altered so as to code for a peptide having properties that are different than those of the naturally occurring peptide. Methods of altering the DNA sequences include but are not limited to site directed mutagenesis. Examples of altered properties include but are not limited to changes in the affinity of an enzyme for a substrate or a receptor for a ligand.

As used herein, “purified” and “isolated” are utilized interchangeably to stand for the proposition that the nucleic acid, protein, or respective fragment thereof in question has been substantially removed from its in vivo environment so that it may be manipulated by the skilled artisan, such as but not limited to nucleotide sequencing, restriction digestion, site-directed mutagenesis, and subcloning into expression vectors for a nucleic acid fragment as well as obtaining the protein or protein fragment in pure quantities so as to afford the opportunity to generate polyclonal antibodies, monoclonal antibodies, amino acid sequencing, and peptide digestion. Therefore, the nucleic acids claimed herein may be present in whole cells or in cell lysates or in a partially purified or substantially purified form. A nucleic acid is considered substantially purified when it is purified away from environmental contaminants. Thus, a nucleic acid sequence isolated from cells is considered to be substantially purified when purified from cellular components by standard methods while a chemically synthesized nucleic acid sequence is considered to be substantially purified when purified from its chemical precursors.

A preferred aspect of the present invention is disclosed in FIGS. 1A-D and SEQ ID NO:1, a rat cDNA encoding an optimized receptor type tyrosine kinase gene, KDR, disclosed as follows:

| | | | | | |
|---|---|---|---|---|---|
| GACCGAGAAA | GCATCTGTGC | CCAGCGCGAG | GTGCAGGATG | GAGAGCAGGG | (SEQ ID NO:1) |
| CGCTGCTAGC | TGTCGCTCTG | TGGTTCTGCG | TGGAGACCCG | AGCCGCCTCT | |
| GTGGGTTTGC | CTGGCGATTC | GCTCCATCCA | CCCAAGCTCA | GCACACAAAA | |
| AGACATACTT | ACAATTTTGG | CAAATACAAC | CCTTCAGATT | ACTTGCAGGG | |
| GACAGAGGGA | CCTGGATTGG | CTTTGGCCCA | ACACTCCGCG | TGACTCTGAG | |
| GAAAGGGTGT | TGGTGACTGA | GTGTGGCGAC | AGTATCTTCT | GCAAGACACT | |
| CACAGTTCCC | AGAGTGGTTG | GAAATGATAC | TGGAGCCTAC | AAGTGCTTCT | |
| ATCGGGACAC | CGATGTCTCC | TCCATCGTTT | ATGTCTATGT | TCAAGATCAC | |
| AGGTCACCAT | TCATCGCCTC | TGTCAGTGAC | GAGCATGGCA | TCGTGTACAT | |
| CACTGAGAAC | AAGAACAAAA | CTGTGGTGAT | CCCATGCCGA | GGGTCGATTT | |
| CAAACCTCAA | CGTGTCACTT | TGTGCTAGGT | ATCCAGAAAA | GAGATTTGTT | |
| CCGGATGGAA | ACAGAATTTC | CTGGGACAGC | GAGAAAGGCT | TTAGTATCCC | |
| CAGTTACATG | ATCAGCTATG | CCGGCATGGT | CTTCTGTGAG | GCAAAGATTA | |
| ATGATGAAAC | GTATCAGTCT | ATCATGTACA | TAGTTCTGGT | TGTAGGATAT | |
| AGGATTTATG | ATGTGGTCCT | GAGCCCCCCT | CATGAAATTG | AGCTATCTGC | |
| CGGAGAAAAG | CTTGTCTTAA | ATTGTACAGC | AAGAACAGAG | CTCAACGTGG | |
| GGCTTGATTT | CAGCTGGCAA | TTCCCGTCCT | CAAAGCATCA | GCATAAGAAG | |
| ATTGTAAACC | GGGATGTGAA | ATCCCTTCCT | GGGAGTGTGG | CAAAGATGTT | |
| TTTGAGCACC | TTGACCATAG | ACAGTGTGAC | CAAGAGTGAC | CAAGGAGAAT | |
| ACACCTGCAC | AGCGTACAGT | GGACTGATGA | CCAAGAAAAA | TAAAACATTT | |
| GTCCGAGTTC | ATACAAAACC | TTTTATTGCT | TTTGGTAGCG | GGATGAAATC | |
| TTTGGTGGAA | GCCACTGTGG | GCAGCCAAGT | CCGAATCCCT | GTGAAGTATC | |
| TCAGTTACCC | AGCTCCTGAT | ATCAAATGGT | ACAGAAATGG | ACGACCCATT | |
| GAGTCCAATT | ACACAATGAT | CGTTGGTGAT | GAACTCACCA | TCATGGAAGT | |
| GAGTGAAAGA | GATGCGGGAA | ACTACACGGT | CATCCTCACC | AATCCCATTT | |
| CAATGGAGAA | ACAGAGCCAC | ATGGTCTCTC | TGGTTGTGAA | TGTTCGACCC | |
| CAGATCGGTG | AGAAAGCCTT | GATCTCTCCT | ATGGATTCCT | ACCAGTATGG | |
| CACCATGCAG | ACGCTGACAT | GCACAGTCTA | TGCCAACCCT | CCCCTGCACC | |
| ACATCCAATG | GTACTGGCAG | CTAGAAGAAG | CATGCTCCTA | CAGGCCCAGC | |
| CAAACAAACC | CATATACTTG | TAAAGAATGG | AGACACGTGA | AGGATTTCCA | |
| GGGGGGAAAT | AAGATCGAAG | TCACCAAAAA | CCAATATGCC | CTAATTGAAG | |
| GAAAAAACAA | AACTGTAAGT | ACTCTGGTCA | TCCAGGCTGC | CAACGTGTCC | |
| GCATTATACA | AATGTGAAGC | CATCAACAAA | GCAGGACGAG | GAGAGAGGGT | |
| CATCTCCTTC | CATGTGATCA | GGGGTCCTGA | AATTACTGTC | CAGCCTGCTA | |
| CCCAGCCAAC | CGAGCAGGAG | AGTGTGTCTC | TATTGTGCAC | TGCAGATAGA | |
| AACACGTTTG | AGAACCTCAC | GTGGTACAAG | CTTGGCTCAC | AGGCAACATC | |
| GGTCCACATG | GGCGAATCAC | TCACACCAGT | TTGCAAGAAC | TTGGACGCTC | |
| TTTGGAAACT | GAATGGCACC | GTGTTTTCTA | ACAGCACAAA | CGACATCTTG | |
| ATTGTGGCAT | TCCAGAATGC | CTCCGTGCAG | GACCAAGGCA | ACTATGTCTG | |
| CTCTGCTCAA | GACAAGAAGA | CCAAGAAAAG | ACATTGCCTA | GTCAAGCAGC | |
| TCGTCATCCT | AGAGCGCATG | GCACCCATGA | TCACTGGAAA | TCTGGAGAAT | |
| CAGACAACAA | CCATTGGTGA | GACCATCGAA | GTTGTTTGTC | CAACATCTGG | |
| AAACCCTACC | CCCCTCATTA | CATGGTTCAA | AGACAATGAG | ACCCTTGTAG | |
| AAGATTCAGG | CATTGTACTA | AAAGACGGGA | ACCGGAACCT | AACTATCCGA | |
| AGGGTGAGGA | AGGAAGACGG | GGGGCTCTAC | ACCTGCCAGG | CCTGCAATGT | |
| CCTTGGCTGT | GCAAGAGCAG | AGACACTCTT | CATAATAGAA | GGTGCCCAGG | |
| AAAAGACCAA | CTTGGAAGTC | ATTATTCTCG | TCGGCACTGC | AGTGATCGCC | |
| ATGTTCTTCT | GGCTACTTCT | TGTCATTGTT | CTACGGACCG | TTAAGCGGGC | |
| CAATGAAGGG | GAACTGAAGA | CAGGCTACTT | GTCCATTGTC | ATGGATCCAG | |
| ATGAACTGCC | CTTGGATGAG | CGCTGTGAAC | GCTTGCCTTA | TGATGCCAGC | |
| AAGTGGGAGT | TCCCCAGGGA | CCGGCTGAAA | CTAGGAAAAC | CTCTTGGCCG | |
| TGGTGCCTTT | GGCCAAGTGA | TTGAGGCAGA | TGCCTTTGGA | ATCGAGAAGA | |
| CAGCGACTTG | CAAAACAGTG | GCTGTCAAGA | TGTTGAAAGA | GGGAGCAACA | |
| CACAGCGAGC | ACCGAGCCCT | CATGTCCGAA | CTCAAGATCC | TCATCCACAT | |
| TGGCCACCAT | CTCAATGTGG | TGAACCTGCT | GGGTGCCTGC | ACGAAGCCCG | |
| GAGGGCCTCT | CATGGTGATT | GTAGAATTCT | GCAAGTTTGG | AAACCTATCA | |
| ACTTACTTAC | GGGGCAAGAG | AAATGAATTC | GTGCCCTATA | AGAGCAAAGG | |
| GGCACGCTTC | CGCTCTGGGA | AAGACTATGT | TGGGGAGCTC | TCCGTAGACC | |
| TGAAGCGGCG | CTTGGACAGC | ATCACCAGCA | GTCAGAGCTC | TGCCAGCTCA | |
| GGTTTTGTGG | AGGAGAAATC | CCTCAGTGAC | GTAGAGGAAG | AAGAAGCTTC | |
| TGAAGAACTC | TACAAGGACT | TCCTGACCTT | GGAGCATCTC | ATCTGTTACA | |
| GCTTCCAAGT | GGCTAAGGGC | ATGGAGTTCT | TGGCATCAAG | GAAGTGTATC | |
| CACAGGGACC | TGGCAGCACG | AAACATTCTC | CTATCGGAGA | AGAACGTGGT | |
| TAAGATCTGT | GACTTTGGCT | TGGCCCGGGA | CATTTATAAA | GACCCAGATT | |
| ACGTCAGAAA | AGGAGATGCC | CGACTCCCTT | TGAAGTGGAT | GGCTCCGGAA | |
| ACAATTTTTG | ACAGAGTATA | CACAATTCAG | AGTGACGTGT | GGTCTTTTGG | |
| TGTTTTGCTC | TGGGAAATAT | TTTCCTTAGG | TGCTTCCCCA | TATCCTGGGG | |
| TCAAGATTGA | TGAAGAATTT | TGTAGGAGAT | TGAAAGAAGG | AACGAGAATG | |
| CGGGCTCCTG | ACTACACCAC | CCCAGAAATG | TACCAAACCA | TGCTGGATTG | |
| CTGGCATGAG | GACCCCAACC | AGAGACCCGC | GTTTTCAGAG | TTGGTGGAGC | |
| ACTTGGGAAA | TCTCCTGCAA | GCAAATGCTC | AGCAGGATGG | CAAAGACTAT | |
| ATTGTTCTTC | CAATGTCAGA | GACACTGAGC | ATGGAAGAGG | ATTCTGGACT | |
| CTCCCTGCCT | ACCTCACCTG | TTTCCTGTAT | GGAGGAAGAG | GAAGTGTGCG | |
| ACCCCAAATT | CCATTATGAC | AACACAGCAG | GAATCAGTGA | TTATCTGCAG | |
| AACAGCAAGC | GAAAAAGCCG | GCCAGTGAGT | GTAAAAACAT | TTGAAGATAT | |
| CCCTTTGGAG | GAACCAGAAG | TAAAAGTGAT | TCCAGATGAC | AGCCAGACAG | |
| ACAGTGGGAT | GGTCCTTGCC | TCAGAAGAGC | TGAAAACTCT | GGAAGACAGG | |
| AACAAATTAT | CTCCATCTTT | TGGTGGGATG | ATGCCCAGTA | AAAGCAGGGA | |
| GTCTGTGGCC | TCGGAAGGCT | CCAACCAGAC | CAGCGGCTAC | CAGTCTGGGT | |
| ATCACTCAGA | CGACACAGAT | ACCACCGTGT | ACTCCAGCGA | CGAGGCAGGA | |
| CTTTTAAAGC | TGGTGGATGT | TGCAGGGCAC | GTTGACTCTG | GGACCACACT | |
| GCGCTCATCT | CCTGTTTAAA | AGGAAGTGGC | CCTGTCCCGT | CCCCGCCCCC | |
| AACTCCTGGA | AATAACTCGA | GAGGTGCTGC | TTAGATTTTC | AAGTGTTGTT | |
| CTTTCCACCA | CTCGGAAGTA | GCCGCATTTG | ATTTTCATTT | CAGAAGAGGG | |
| ACCTCAGACG | GCAAGAAGCT | TGTCCTCAGG | GCATTTCCAG | AAAAATGCCC | |
| ATGACCCAAG | AATGTGTTGA | CTATACTCTC | TTTTCCATTG | GTTTAAAAAT | |
| CCTATATATT | GTGCCCTGCT | GCGGGTCTCA | CTACCAGTTA | AAACAAAAGA | |
| CGTTCAAACA | GCGGCTCTAT | CCTCCAAGAA | GTAGCCATAC | CCAGGCAATG | |
| GAGCCCTCTG | TGAAACTGGA | TAAAATGGGC | GATGTTAGTG | CTTTGTGTGT | |
| TGGGATGGGT | GAGATGTCCC | AGGGCTGAGT | CTACCTAAAA | GGCTTTGTGG | |
| AGGATGTGGG | CTATGAGCCA | AGTGTTAAGT | GTGAGATGTG | GACTGGTAGG | |
| AAGGAAGGAG | CAAGCTCGCT | CAGAGAGCGG | TTGGAGCCTG | CAGATGCATT | |
| GTGCTGGCTG | TGGTGGAGGT | GAGCATGTGG | CCTGTCAGGA | AACGCCAAGG | |
| CGGCTGTCGG | GGTTTGGTTT | TGGAAGGTTG | CGTGCTCTTC | ACGGTTGGGC | |
| TACAGGCGAG | TTCCCTGTGC | TGTTTCCTAC | TCCTAATGAG | AGTTCCTTCC | |
| GGACTCTTAC | GTGTCTCCTG | GCCTAGCCCC | AGGAAGGAAA | TGACGCAGCT | |
| TGCTCCTCAT | CTCCCAGGCT | GTGCCTTAAC | TCAGAATACT | AAAAGAGAGG | |
| GACTTTGGCC | GAGGCTCCGC | TCCTTGTCAT | GCTGAAGAAC | TGTGAGAACA | |
| CAACAGAAAC | TCAGGGTTTC | TGCTGGGTGG | ATACCCACTT | GTCTGCCCTG | |
| GTGGCAGTGT | CTGAGGGTTT | TGTCAAGTGG | CGATGGTAAA | GGCTCAGACA | |
| GGATGTATCC | CTTTGTTCTT | CCTCTAACTC | CACTTCTGTC | TTGCCACACC | |
| CCCCCCTCCC | CAGTGCTCAG | TATTTTAGCT | TTGTGGCCAC | GTGATGGCAG | |
| AAGGTCTTAA | TTGGTTGGTT | TTGCTCTCCA | GATAAAATCA | CTAGTCAGAT | |
| TTCGAAATTA | CTTTATAGCC | AAGGTCTGAT | AACATCTACT | GTATCGTTTA | |
| GAATTTAACA | TATAAAGCTG | TGTCTACTGG | TTTTTTTTTT | TTTTGCCCTT | |
| GGGCATATGT | TTTTCAAAAG | AGAAACTACT | TTTCATTTGG | TACCATAGCG | |
| TGACGAGCAG | GGGCCAATGA | CTGTAAAACA | TGCTGTGGCA | CATATATTTA | |
| TAGTCTGTTA | TGTGGAACAA | ATGTAATATA | TTGAAACTTT | ATATTATATA | |
| TAAGGAAGTT | TGTACTATCC | GCATTTCGTA | TCAGTATTAT | GTAGCATGAC | |
| AGAGACTGTG | AGGTCTGAGC | AGCTGGTGGC | TCAGGACGTT | GAGAAACTCG | |
| AAGGAATCCT | TTCGTGAGGA | TGCGCAGCTA | TCCCTACCCA | TCTCTCTCAC | |
| CTCAAACGGA | GGAGAAAGGG | GAATCAGAGA | TAATGTGAGT | GTGTCCTTGT | |
| TCTCTGTTCT | TAGGAGGAAT | GTTCTTACCA | ACTGTTCATA | CGCTTTATAA | |
| ACCAATAAAT | GTATTCTGAG | TAAAGAAAAA | AAAAAAAAAA | AAA. | |

The present invention also relates to recombinant vectors and recombinant hosts, both prokaryotic and eukaryotic, which contain the substantially purified nucleic acid molecules disclosed throughout this specification.

The present invention relates to a purified form of an optimized rat receptor type tyrosine kinase protein, KDR, a receptor tyrosine kinase expressed on rat endothelial cells.

The present invention further relates to a purified form of a rat receptor type tyrosine kinase protein, KDR, comprising an amino acid sequence retaining Asp at position 1083, and alternatively retaining Asp at position 1083 in combination with Ala at position 1061, Val at position 1077, and/or Glu at position 1110.

The present invention also relates to a purified form of a rat receptor type tyrosine kinase protein, KDR, comprising or consisting of the amino acid sequence as disclosed in FIG. 2 and as set forth in SEQ ID NO:2.

A preferred aspect of the present invention is a purified form of the receptor type tyrosine kinase protein, KDR, a rat KDR protein which includes Asn at position 519, Gln at position 560, Val at position 563, Ala at position 753, Val at position 781, Leu at position 782, Asp at position 1083, Ala at position 1061, Val at position 1077 and Glu at position 1110, as disclosed below. The amino acid differences of the optimized rat KDR of the present invention when compared to the published rat KDR sequence are underlined.

| | | | | | |
|---|---|---|---|---|---|
| MESRALLAVA | LWFCVETRAA | SVGLPGDSLH | PPKLSTQKDI | LTILANTTLQ | (SEQ ID NO:2) |
| ITCRGQRDLD | WLWPNTPRDS | EERVLVTECG | DSIFCKTLTV | PRVVGNDTGA | |
| YKCFYRDTDV | SSIVYVYVQD | HRSPFIASVS | DEHGIVYITE | NKNKTVVIPC | |
| RGSISNLNVS | LCARYPEKRF | VPDGNRISWD | SEKGFTIPSY | MISYAGMVFC | |
| EAKINDETYQ | SIMYIVLVVG | YRIYDVVLSP | PHEIELSAGE | KLVLNCTART | |
| ELNVGLDFSW | QFPSSKHQHK | KIVNRDVKSL | PGTVAKMFLS | TLTIDSVTKS | |
| DQGEYTCTAY | SGLMTKKNKT | FVRVHTKPFI | AFGSGMKSLV | EATVGSQVRI | |
| PVKYLSYPAP | DIKWYRNGRP | IESNYTMIVG | DELTIMEVSE | RDAGNYTVIL | |
| TNPISMEKQS | HMVSLVVNVP | PQIGEKALIS | PMDSYQYGTM | QTLTCTVYAN | |
| PPLHHIQWYW | QLEEACSYRP | SQTNPYTCKE | WRHVKDFQGG | NKIEVTKNQY | |
| ALIEGKNKTV | STLVIQAANV | SALYKCEAIN | KAGRGERVIS | FHVIRGPEIT | |
| VQPATQPTEQ | ESVSLLCTAD | RNTFENLTWY | KLGSQATSVH | MGESLTPVCK | |
| NLDALWKLNG | TVFSNSTNDI | LIVAFQNASL | QDQGNYVCSA | QDKKTKKRHC | |
| LVKQLVILER | MAPMITGNLE | NQTTTIGETI | EVVCPTSGNP | TPLITWFKDN | |
| ETLVEDSGIV | LKDGNRNLTI | RRVRKEDGGL | YTCQACNVLG | CARAETLFII | |
| EGAQEKTNLE | VIILVGTAVI | AMFFWLLLVI | VLRTVKRANE | GELKTGYLSI | |
| VMDPDELPLD | ERCERLPYDA | SKWEFPRDRL | KLGKPLGRGA | FGQVIEADAF | |
| GIDKTATCKT | VAVKMLKEGA | THSEHRALMS | ELKILIHIGH | HLNVVNLLGA | |
| CTKPGGPLMV | IVEFCKFGNL | STYLRGKRNE | FVPYKSKGAR | FRSGKDYVGE | |
| LSVDLKRRLD | SITSSQSSAS | SGFVEEKSLS | DVEEEEASEE | LYKDFLTLEH | |
| LICYSFQVAK | GMEFLASRKC | IHRDLAARNI | LLSEKNVVKI | CDFGLARDIY | |
| KDPDYVRKGD | ARLPLKWMAP | ETIFDRVYTI | QSDVWSFGVL | LWEIFSLGAS | |
| PYPGVKIDEE | FCRRLKEGTR | MRAPDYTTPE | MYQTMLDCWH | EDPNQRPAFS | |
| ELVEHLGNLL | QANAQQDGKD | YIVLPMSETL | SMEEDSGLSL | PTSPVSCMEE | |
| EEVCDPKFHY | DNTAGISHYL | QNSKRKSRPV | SVKTFEDIPL | EEPEVKVIPD | |
| DSQTDSGMVL | ASEELKTLED | RNKLSPSFGG | MMPSKSRESV | ASEGSNQTSG | |
| YQSGYHSDDT | DTTVYSSDEA | GLLKLVDVAG | HVDSGTTLRS | SPV. | |

The present invention also relates to biologically active fragments and/or mutants of the KDR protein as initially set forth as SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists for KDR function.

The present invention also relates to subcellular membrane fractions of the recombinant host cells (both prokaryotic and eukaryotic as well as both stably and transiently transformed cells) comprising the nucleic acids of the present invention. These subcellular membrane fractions will comprise wild-type or rat mutant forms of KDR at levels substantially above wild-type levels and hence will be useful in various assays described throughout this specification.

Therefore, the present invention relates to methods of expressing the receptor type tyrosine kinase gene, KDR, and biological equivalents disclosed herein, assays employing these receptor type tyrosine kinase genes, cells expressing these receptor type tyrosine kinase genes, and agonistic and/or antagonistic compounds identified through the use of these receptor type tyrosine kinase genes and expressed rat KDR protein, including, but not limited to, one or more modulators of the rat KDR-dependent kinase through direct contact with the kinase domain of rat KDR or a compound which prevents binding of VEGF to rat KDR, or either prevents or promotes receptor dimerization and/or activation thereby either inducing or antagonizing transduction of the normal intracellular signals associated with VEGF-induced angiogenesis

As used herein, a “biologically active equivalent” or “functional derivative” of a wild-type rat KDR possesses a biological activity that is substantially similar to the biological activity of the wild type rat KDR. The term “functional derivative” is intended to include the “fragments,” “mutants,” “variants,” “degenerate variants,” “analogs” and “homologues” or to “chemical derivatives” of the wild type rat KDR protein. The term “fragment” is meant to refer to any polypeptide subset of wild-type rat KDR. The term “mutant” is meant to refer to a molecule that may be substantially similar to the wild-type form but possesses distinguishing biological characteristics. Such altered characteristics include but are in no way limited to altered substrate binding, altered substrate affinity and altered sensitivity to chemical compounds affecting biological activity of the rat KDR or rat KDR functional derivative. The term “variant” is meant to refer to a molecule substantially similar in structure and function to either the entire wild-type protein or to a fragment thereof. A molecule is “substantially similar” to a wild-type rat KDR-like protein if both molecules have substantially similar structures or if both molecules possess similar biological activity. Therefore, if the two molecules possess substantially similar activity, they are considered to be variants even if the structure of one of the molecules is not found in the other or even if the two amino acid sequences are not identical. The term “analog” refers to a molecule substantially similar in function to either the full-length rat KDR protein or to a biologically active fragment thereof.

Any of a variety of procedures may be used to clone rat KDR. These methods include, but are not limited to, (1) a RACE PCR cloning technique (Frohman, et al., 1988, Proc. NatL. Acad. Sci. USA 85: 8998-9002). 5′ and/or 3′ RACE may be performed to generate a full-length cDNA sequence. This strategy involves using gene-specific oligonucleotide primers for PCR amplification of rat KDR cDNA. These gene-specific primers are designed through identification of an expressed sequence tag (EST) nucleotide sequence which has been identified by searching any number of publicly available nucleic acid and protein databases; (2) direct functional expression of the rat KDR cDNA following the construction of a rat KDR-containing cDNA library in an appropriate expression vector system; (3) screening a rat KDR-containing cDNA library constructed in a bacteriophage or plasmid shuttle vector with a labeled degenerate oligonucleotide probe designed from the amino acid sequence of the rat KDR protein; (4) screening a rat KDR-containing cDNA library constructed in a bacteriophage or plasmid shuttle vector with a partial cDNA encoding the rat KDR protein. This partial cDNA is obtained by the specific PCR amplification of rat KDR DNA fragments through the design of degenerate oligonucleotide primers from the amino acid sequence known for other kinases which are related to the rat KDR protein; (5) screening a rat KDR-containing cDNA library constructed in a bacteriophage or plasmid shuttle vector with a partial cDNA encoding the human KDR protein. This strategy may also involve using gene-specific oligonucleotide primers for PCR amplification of rat KDR cDNA identified as an EST as described above; or (6) designing 5′ and 3′ gene specific oligonucleotides using SEQ ID NO:1 as a template so that either the full-length cDNA may be generated by known RACE techniques, or a portion of the coding region may be generated by these same known RACE techniques to generate and isolate a portion of the coding region to use as a probe to screen one of numerous types of cDNA and/or genomic libraries in order to isolate a full-length version of the nucleotide sequence encoding rat KDR.

It is readily apparent to those skilled in the art that other types of libraries, as well as libraries constructed from other cell types-or species types, may be useful for isolating a rat KDR-encoding DNA or a rat KDR homologue. Other types of libraries include, but are not limited to, cDNA libraries derived from other cells or cell lines other than rat cells or tissue such as murine cells, rodent cells or any other such vertebrate host which may contain rat KDR-encoding DNA. Additionally a rat KDR gene and homologues may be isolated by oligonucleotide- or polynucleotide-based hybridization screening of a vertebrate genomic library, including but not limited to, a murine genomic library, a rodent genomic library, as well as concomitant rat genomic DNA libraries.

It is readily apparent to those skilled in the art that suitable cDNA libraries may be prepared from cells or cell lines which have KDR activity. The selection of cells or cell lines for use in preparing a cDNA library to isolate a cDNA encoding rat KDR may be done by first measuring cell-associated KDR activity using any known assay available for such a purpose.

Preparation of cDNA libraries can be performed by standard techniques well known in the art. Well known cDNA library construction techniques can be found for example, in Sambrook et al., 1989, Molecular Cloning: A Laboratory Manual; Cold Spring Harbor Laboratory, Cold Spring Harbor, N.Y. Complementary DNA libraries may also be obtained from numerous commercial sources, including but not limited to Clontech Laboratories, Inc. and Stratagene.

It is also readily apparent to those skilled in the art that DNA encoding rat KDR may also be isolated from a suitable genomic DNA library. Construction of genomic DNA libraries can be performed by standard techniques well known in the art. Well known genomic DNA library construction techniques can be found in Sambrook, et al., supra.

In order to clone the rat KDR gene by one of the preferred methods, the amino acid sequence or DNA sequence of rat KDR or a homologous protein may be necessary. To accomplish this, the KDR protein or a homologous protein may be purified and partial amino acid sequence determined by automated sequenators. It is not necessary to determine the entire amino acid sequence, but the linear sequence of two regions of 6 to 8 amino acids can be determined for the PCR amplification of a partial rat KDR DNA fragment. Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the rat KDR sequence but others in the set will be capable of hybridizing to rat KDR DNA even in the presence of DNA oligonucleotides with mismatches. The mismatched DNA oligonucleotides may still sufficiently hybridize to the rat KDR DNA to permit identification and isolation of rat KDR encoding DNA. Alternatively, the nucleotide sequence of a region of an expressed sequence may be identified by searching one or more available genomic databases. Gene-specific primers may be used to perform PCR amplification of a cDNA of interest from either a cDNA library or a population of cDNAs. As noted above, the appropriate nucleotide sequence for use in a PCR-based method may be obtained from SEQ ID NO:1, either for the purpose of isolating overlapping 5′ and 3′ RACE products for generation of a full-length sequence coding for rat KDR, or to isolate a portion of the nucleotide sequence coding for rat KDR for use as a probe to screen one or more cDNA- or genomic-based libraries to isolate a full-length sequence encoding rat KDR or rat KDR-like proteins.

It is also readily apparent to those skilled in the art that DNA encoding rat KDR may be synthetically generated. Many different methods are used for assembling and generating synthetic genes. In one such method, a series of sequentially overlapping oligonucleotides are synthesized. The oligonucleotides anneal to form a double stranded DNA fragment containing nicks on both strands. DNA ligase, an enzyme that catalyses the formation of phosphodiester bonds between the 5′-phosphate of one double-strand oligonucleotide fragment and the 3′-hydroxl terminus on another adjacent double-strand oligonucleotide, is used to seal the nicks. Synthetic genes can also be made using the template-directed and primer-dependent 5′- to 3′-synthesis capabilities of the large subunit of the enzyme DNA-Polymerase I (Klenow fragment). The polymerase uses deoxynucleoside-triphosphates to fill in gaps once end annealing of the long oligonucleotides occurs. Any nick in the resulting double-stranded DNA is sealed by DNA ligase. Finally, very long oligonucleotide chains can be synthesized so that their 3′-ends overlap upon annealing. A subsequent filling-in reaction using DNA polymerase completes the full-length, double-stranded DNA. A number of companies specialize in generating synthetic genes with a high degree of sequence accuracy including Entelechon GmbH (Regensburg, Germany) and MCLAB (South San Francisco, Calif.).

In an exemplified method performed by Pangene Corporation (Fremont, Calif.), the rat KDR cDNA of the present invention was generated by screening a rat spleen plasmid cDNA library with two biotinylated targeting probes (A and B). Separate rounds of screening were performed for each probe. Probes A and B were made by PCR from the library DNA. Probe A corresponds to bases 282 to 968 of NM_{—}013062 (rat Flk1, NCBI GenBank database) and was obtained using forward primer, TGGTTCTGCGTGGAGAC (SEQ ID NO:3), and reverse primer, TTCTCCGGCAGATAGCTC (SEQ ID NO:4). Probe B corresponds to bases 2664 to 2940 of NM_{—}013062 and was obtained using forward primer, GAACTGCCCTTGGATGAG (SEQ ID NO:5), and reverse primer, GCAGGTTCACCACATTGA (SEQ ID NO:6). After being denatured, each probe was complexed with recombinase protein(s) such as RecA; and the protein coated probe was mixed with the cDNA library, allowing the probe to interact with homologous sequences and to form triple stranded nucleoprotein complexes. The hybrids that were formed were isolated magnetically, and the recovered plasmids were used to transform competent E. Coli cells. The resulting colonies were screened by PCR using the following screening primers: forward primer CTGCTAGCTGTCGCTCTG (SEQ ID NO:7) and reverse primer TTCTCCGGCAGATAGCTC (SEQ ID NO:4) for colonies obtained with probe A; forward primer CTGCAGTGATTGCCATGT (SEQ ID NO:8) and reverse primer GGGCACGAATTCATTTCT (SEQ ID NO:9) for colonies obtained with probe B. Purified plasmids from colonies that yielded a PCR product were further analyzed by restriction digestion and DNA sequencing.

The cloned rat KDR cDNA obtained through the methods described above may be recombinantly expressed by molecular cloning into an expression vector (such as pcDNA3.neo, pcDNA3.1, pCR2.1, pBlueBacHis2 or pLITMUS28) containing a suitable promoter and other appropriate transcription regulatory elements, and transferred into prokaryotic or eukaryotic host cells to produce recombinant rat KDR. Expression vectors are defined herein as DNA sequences that are required for the transcription of cloned DNA and the translation of their mRNAs in an appropriate host. Such vectors can be used to express eukaryotic DNA in a variety of recombinant host cells such as bacteria, blue green algae, plant cells, insect cells and mammalian cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Methods to determine the rat KDR cDNA sequence(s) that yields optimal levels of rat KDR are well known in the art. Following determination of the rat KDR cDNA cassette yielding optimal expression, this rat KDR cDNA construct is transferred to a variety of expression vectors (including recombinant viruses), including but not limited to those for mammalian cells, plant cells, insect cells, oocytes, bacteria and yeast cells. Techniques for such manipulations can be found described in Sambrook, et al., supra, are well known and available to artisan of ordinary skill in the art. Therefore, another aspect of the present invention includes host cells that have been engineered to contain and/or express DNA sequences encoding rat KDR. An expression vector containing DNA encoding rat KDR protein may be used for expression of rat KDR in a recombinant host cell. Such recombinant host cells can be cultured under suitable conditions to produce rat KDR or a biologically equivalent form. Expression vectors may include, but are not limited to, cloning vectors, modified cloning vectors, specifically designed plasmids or viruses. Commercially available mammalian expression vectors may be suitable for recombinant rat KDR expression. Also, a variety of commercially available bacterial, fungal cell, and insect cell expression vectors may be used to express recombinant rat KDR in the respective cell types.

Recombinant host cells may be prokaryotic or eukaryotic, including but not limited to, bacteria such as E. coli, fungal cells such as yeast, mammalian cells including, but not limited to, cell lines of bovine, porcine, monkey, and rodent origin; and insect cells.

The expression vector may be introduced into host cells via any one of a number of techniques including but not limited to transformation, transfection, protoplast fusion, and electroporation. The expression vector-containing cells are individually analyzed to determine whether they produce rat KDR protein. Identification of rat KDR expressing cells may be done by several means, including but not limited to immunological reactivity with anti-rat KDR antibodies, labeled ligand binding and the presence of host cell-associated rat KDR activity.

Expression of rat KDR DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

Levels of rat KDR in host cells is quantified by a variety of techniques including, but not limited to, immunoaffinity and/or ligand affinity techniques. KDR-specific affinity beads or KDR-specific antibodies are used to isolate ³⁵S-methionine labeled or unlabelled KDR. Labeled KDR protein is analyzed by SDS-PAGE. Unlabelled KDR protein is detected by Western blotting, ELISA or RIA assays employing either KDR protein specific antibodies and/or antiphosphotyrosine antibodies.

Following expression of KDR in a host cell, KDR protein may be recovered to provide KDR protein in active form. Several KDR protein purification procedures are available and suitable for use. Recombinant KDR protein may be purified from cell lysates and extracts, or from conditioned culture medium, by various combinations of, or individual application of salt fractionation, ion exchange chromatography, size exclusion chromatography, hydroxylapatite adsorption chromatography and hydrophobic interaction chromatography.

In addition, recombinant KDR protein can be separated from other cellular proteins by use of an immunoaffinity column made with monoclonal or polyclonal antibodies specific for full-length KDR protein, or polypeptide fragments of KDR protein. Additionally, polyclonal or monoclonal antibodies may be raised against a synthetic peptide (usually from about 9 to about 25 amino acids in length) from a portion of the protein as disclosed in SEQ ID NO:2. Monospecific antibodies to rat KDR are purified from mammalian antisera containing antibodies reactive against rat KDR or are prepared as monoclonal antibodies reactive with rat KDR using the technique of Kohler and Milstein (1975, Nature 256: 495-497).

Monospecific antibody as used herein is defined as a single antibody species or multiple antibody species with homogenous binding characteristics for rat KDR. Homogenous binding as used herein refers to the ability of the antibody species to bind to a specific antigen or epitope, such as those associated with rat KDR, as described above. Rat KDR-specific antibodies are raised by immunizing animals such as mice, guinea pigs, rabbits, goats, horses and the like, with an appropriate concentration of rat KDR protein or a synthetic peptide generated from a portion of rat KDR with or without an immune adjuvant. Preimmune serum is collected prior to the first immunization. Each animal receives between about 0.1 mg and about 1000 mg of rat KDR protein associated with an acceptable immune adjuvant, including but not limited to, Freund's complete, Freund's incomplete, alum-precipitate, water in oil emulsion containing Corynebacterium parvum and tRNA. The initial immunization consists of rat KDR protein or a peptide fragment thereof in, preferably, Freund's complete adjuvant at multiple sites either subcutaneously (SC), intraperitoneally (IP) or both. The animals may or may not receive booster injections following the initial immunization depending on determination of antibody titer. At about 7 days after each booster immunization, or about weekly after a single immunization, the animals are bled, the serum collected, and aliquots are stored at about −20° C.

Monoclonal antibodies (mAb) reactive with rat KDR protein are prepared by immunizing inbred mice, preferably Balb/c, with rat KDR protein. The mice are immunized by the IP or SC route with about 1 mg to about 100 mg, preferably about 10 mg, of rat KDR protein in about 0.5 ml buffer or saline incorporated in an equal volume of an acceptable adjuvant, as discussed above. Immunized mice are given one or more booster immunizations by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. The antibody producing cells and myeloma cells are fused in polyethylene glycol. Fused hybridoma cells are selected by growth in hypoxanthine, thymidine and aminopterin supplemented Dulbecco's Modified Eagles Medium (DMEM) by procedures known in the art. Supernatant fluids are collected form growth positive wells and are screened for antibody production by an immunoassay such as solid phase immunoradioassay (SPIRA) using rat KDR as the antigen. The culture fluids are also tested in the Ouchterlony precipitation assay to determine the isotype of the mAb. Hybridoma cells from antibody positive wells are cloned by a technique such as the soft agar technique of MacPherson, 1973, Soft Agar Techniques, in Tissue Culture Methods and Applications, Kruse and Paterson, Eds., Academic Press.

Monoclonal antibodies are produced in vivo by injection of pristine primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of anti-rat KDR mAb is carried out by growing the hybridoma in DMEM containing about 2% fetal calf serum to obtain sufficient quantities of the specific mAb. The mAb are purified by techniques known in the art.

Antibody titers of ascites or hybridoma culture fluids are determined by various serological or immunological assays known in the art. Similar assays are used to detect the presence of rat KDR in fluids or tissue and cell extracts.

It is readily apparent to those skilled in the art that the above described methods for producing monospecific antibodies may be utilized to produce antibodies specific for a rat KDR peptide fragments, or a respective a full-length rat KDR.

The rat KDR protein of the present invention is suitable for use in an assay procedure for the identification of compounds which modulate KDR activity. A KDR-containing fusion construct, such as a GST-KDR fusion as discussed within this specification, is useful to measure KDR activity. Kinase activity can be measured, for example, using a modified version of the homogeneous time-resolved tyrosine kinase assay described by Park et al. (1999, Anal. Biochem. 269:94-104).

Soluble recombinant GST-kinase domain fusion proteins are expressed in a baculovirus system (Pharmingen) according to a protocol recommended by the manufacturer. The KDR sequence is subcloned into a baculovirus expression vector (pGcGHLT-A, Pharmingen) containing an in frame 6× histidine tag and a GST tag, and the resulting vector is expressed in Sf9 insect cells. After confirming expression of GST-KDR, a high titer recombinant baculovirus stock is produced, expression conditions are optimized, and a scaled up expression of rat KDR-GST fusion is performed. The KDR fusions are then purified from the Sf9 cell lysate by affinity chromatography. First, about 30 grams of frozen Sf9 cell pellets are lysed in 4 volumes of lysis buffer containing 0.5% NP40, 1% Triton X-100, 135 mM NaCl, 1.5 mM H₂NaPO₄, 4.3 mM HNa₂PO₄, and COMPLETE™ protease inhibitor cocktail (Roche). After centrifugation at 40,000 RPM for 20 minutes, the supernatant is loaded onto a 5-ml GSTrap column (AmershamPharmacia) pre-equilibrated with lysis buffer. The column is washed exhaustively with lysis buffer, and subsequently, with phosphate-buffered saline (PBS) containing protease inhibitors. Bound proteins are eluted with 10 mM glutathione in 50 mM Tris-HCl (pH 8.0). The eluted protein fractions are buffer-exchanged into Ni-NTA Binding Buffer (50 mM NaH₂PO₄, 300 mM NaCl, 10 mM imidazole, pH 8.0) using a Sephadex G-25 desalting column, and loaded onto a Ni-NTA Superflow (Qiagen) column pre-equilibrated with the same buffer. The Ni-NTA column is washed exhaustively with Ni-NTA Binding Buffer followed by Ni-NTA Wash Buffer (50 mM NaH₂PO₄, 300 mM NaCl, 20 mM imidazole, pH=8.0). The bound protein(s) are eluted with Ni-NTA Elution Buffer (50 mM NaH₂PO₄, 300 mM NaCl, 250 mM imidazole, pH 8.0). The eluted protein fractions are pooled and dialyzed against 50% glycerol, 2 mM DTT, 50 MM Tris-HCl (pH 7.4). The protein concentrations of the dialyzed fusion proteins are determined using Coomassie Plus Protein Assay (Pierce) with BSA as standard.

The KDR kinase assay comprises the following steps:

1. Prepare a master reaction mix containing 0.83 μM substrate (biotinylated EQEDEPEGDYFEWLE; SEQ ID NO:10), 8.3 μM ATP, 10 mM MgCl₂, 2 mM MnCl₂, 100 mM NaCl, 50 mM Tris-HCl (pH 7.2), 0.5 mg/ml BSA, 0.5 mM Na₃VO₄, and 0.5 mM TCEP.

2. Distribute 50 μl of the master reaction mix to wells of a black 96-well plate.

3. Initiate the kinase reactions with the addition of 10 μl of GST-tagged KDR (wild type or mutant) pre-serial-diluted in the reaction mix buffer less the substrate and ATP. Final concentration of GST-KDR in the reaction if from 0 to 169 nM, achieved by serial dilutions.

4. Allow the reaction to proceed for 35 minutes at room temperature with shaking.

5. Stop by addition of 50 μl of a quench buffer containing 0.8 μg/ml Eu(K)-PT-66 (an europium cryptate-labeled anti-phosphotyrosine antibody), 10 μg/ml streptavidin-XL665, 100 mM EDTA, 0.5 mM KF, and 0.1% Triton X-100.

6. Incubate the quenched reactions for 5 hours at room temperature.

7. Read in a Discovery (Packard), a time-resolved fluorescence detector.

The rat KDR protein of the present invention may be obtained from both native and recombinant sources (as a full-length protein, biologically active protein fragment, or fusion construction) for use in an assay procedure to identify rat KDR modulators. Modulating KDR includes the inhibition or activation of the kinase which affects the mitogenic function of VEGF. Compounds which modulate KDR include agonists and antagonists. In general, an assay procedure to identify rat KDR modulators will contain the intracellular domain of rat KDR, and a test compound or sample which contains a putative KDR kinase agonist or antagonist. The test compounds or samples may be tested directly on, for example, purified KDR, KDR kinase or a GST-KDR kinase fusion, subcellular fractions of KDR-producing cells whether native or recombinant, whole cells expressing rat KDR whether native or recombinant, intracellular KDR protein fragments and respective deletion fragments, and/or extracellular KDR protein fragments and respective deletion fragments. The test compound or sample may be added to KDR in the presence or absence of a known rat KDR substrate. The modulating activity of the test compound or sample may be determined by, for example, analyzing the ability of the test compound or sample to bind to the KDR intracellular domain, activate the protein, inhibit the protein, inhibit or enhance the binding of other compounds to rat KDR, modifying VEGF receptor regulation, or modifying kinase activity.

To assay for modulators of rat KDR, the above kinase reaction can be altered as follows. After step 2, a small volume (e.g. 1 μl) of a desired compound or vehicle is added to each well already containing the reaction mix. In step 3, the kinase reaction is initiated by addition of GST-KDR of a fixed concentration (instead of being serial diluted). The final GST-KDR concentration before quenching is 5 nM. The remaining steps are unchanged.

The identification of modulators of rat KDR will be useful in treating various human disease states. For example, vascular growth in or near the retina leads to visual degeneration culminating in blindness. VEGF accounts for most of the angiogenic activity produced in or near the retina in diabetic retinopathy. Ocular VEGF mRNA and protein are elevated by conditions such as retinal vein occlusion in primates and decreased pO₂ levels in mice that lead to neovascularization. Expression of VEGF is also significantly increased in hypoxic regions of animal and human tumors adjacent to areas of necrosis. VEGF contributes to tumor growth in vivo by promoting angiogenesis through its paracrine vascular endothelial cell chemotactic and mitogenic activities. Inhibition of KDR is implicated in pathological neoangiogenesis, and compounds which inhibit the mitogenic activity of VEGF via inhibition of KDR will be useful in the treatment of diseases in which neoangiogenesis is part of the overall pathology, such as diabetic retinal vascularization, various forms of cancer and inflammation which demonstrate high levels of gene and protein expression. Examples of such cancers include cancers of the brain, breast, genitourinary tract, lymphatic system, stomach, intestines including colon, pancreas, prostate, larynx and lung. These include histiocytic lymphoma, lung adenocarcinoma, glioblastoma and small cell lung cancers. Examples of inflammation include rheumatoid arthritis, psoriasis, contact dermatis and hypersensitivity reactions.

The present invention is also directed to methods for screening for compounds which modulate the expression of DNA or RNA encoding a rat KDR protein. Compounds which modulate these activities may be DNA, RNA, peptides, proteins, or non-proteinaceous organic molecules. Compounds may modulate by increasing or attenuating the expression of DNA or RNA encoding rat KDR, or the function of rat KDR. Compounds that modulate the expression of DNA or RNA encoding rat KDR or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample. Kits containing at KDR, antibodies to rat KDR, or modified rat KDR may be prepared by known methods for such uses.

The DNA molecules, RNA molecules, recombinant proteins and antibodies of the present invention may be used to screen and measure levels of rat KDR. The recombinant proteins, DNA molecules, RNA molecules and antibodies lend themselves to the formulation of kits suitable for the detection and typing of rat KDR. Such a kit would comprise a compartmentalized carrier suitable to hold in close confinement at least one container. The carrier would further comprise reagents such as recombinant KDR or anti-KDR antibodies suitable for detecting rat KDR. The carrier may also contain a means for detection such as labeled antigen or enzyme substrates or the like.

Pharmaceutically useful compositions comprising modulators of rat KDR may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, modified rat KDR, or either KDR agonists or antagonists including tyrosine kinase activators or inhibitors.

Therapeutic or diagnostic compositions of the invention are administered to an individual in amounts sufficient to treat or diagnose disorders. The effective amount may vary according to a variety of factors such as the individual's condition, weight, sex and age. Other factors include the mode of administration.

The pharmaceutical compositions may be provided to the individual by a variety of routes such as subcutaneous, topical, oral and intramuscular.

The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

The following examples are provided to illustrate the present invention without, however, limiting the same hereto.

### EXAMPLE 1

Isolation of a cDNA Encoding Rat KDR by PCR-Independent Cloning

Materials—A rat spleen plasmid cDNA library was used for screening.

The biotinylated targeting probes (A and B) were made using the following PCR primers:

| | | |
|---|---|---|
| Probe A: | | |
| Forward | 5′-TGGTTCTGCGTGGAGAC-3′; | (SEQ ID NO:3) |
| Reverse | 5′-TTCTCCGGCAGATAGCTC-3′. | (SEQ ID NO:4) |
| Probe B: | | |
| Forward | 5′-GAACTGCCCTTGGATGAG-3′; | (SEQ ID NO:5) |
| Reverse | 5′-GCAGGTTCACCACATTGA-3′. | (SEQ ID NO:6) |

The screening PCR primers used for colonies obtained with Probe A are as follows:

| | | |
|---|---|---|
| Forward | 5′-CTGCTAGCTGTCGCTCTG-3′; | (SEQ ID NO:7) |
| Reverse | 5′-TTCTCCGGCAGATAGCTC-3′. | (SEQ ID NO:4) |

The screening PCR primers used for colonies obtained with Probe B were as follows:

| | | |
|---|---|---|
| Forward | 5′-CTGCAGTGATTGCCATGT-3′; | (SEQ ID NO:8) |
| Reverse | 5′-GGGCACGAATTCATTTCT-3′. | (SEQ ID NO:9) |

Methods: Gene Cloning—A rat spleen plasmid cDNA library was screened by Pangene Corporation (Fremont, Calif.) using its proprietary homologous recombination technology. Two biotinylated targeting probes (A and B) were made by PCR from the library DNA and were used separately in different rounds of screening. Probe A corresponds to bases 282 to 968 of NM_{—}013062 (rat Flk1, NCBI GenBank database), and Probe B corresponds to bases 2664 to 2940 of NM_{—}013062. Each probe was denatured and complexed with recombinase protein(s) such as RecA. The protein coated probe was mixed with the cDNA library to allow the probe to interact with homologous sequences and form triple stranded nucleoprotein complexes. The hybrids that were formed were then isolated magnetically. The plasmids recovered were used to transform competent E. Coli cells, and the resulting colonies were screened by PCR using screening primers specific for colonies obtained from either Probe A or Probe B. Purified plasmids from colonies that yielded a PCR product were further analyzed by restriction digestion and DNA sequencing.

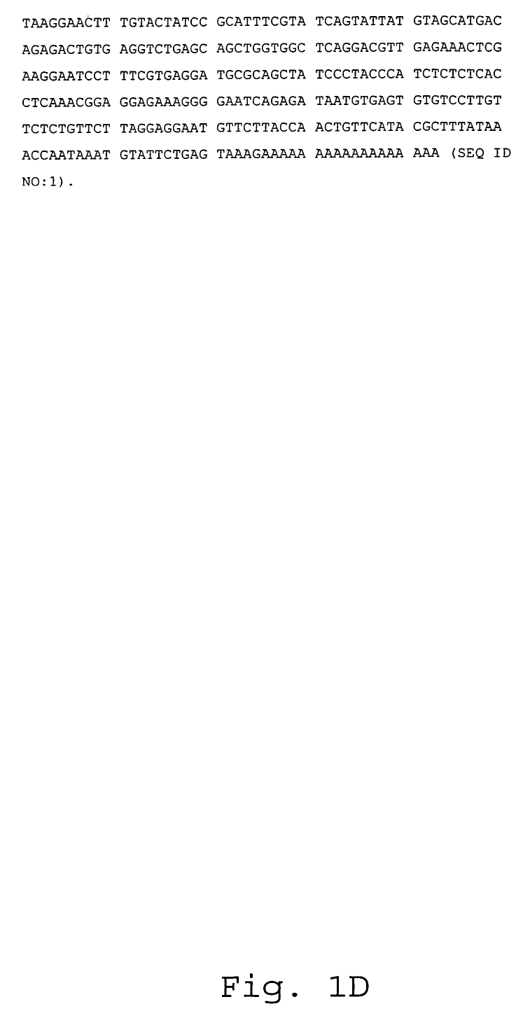

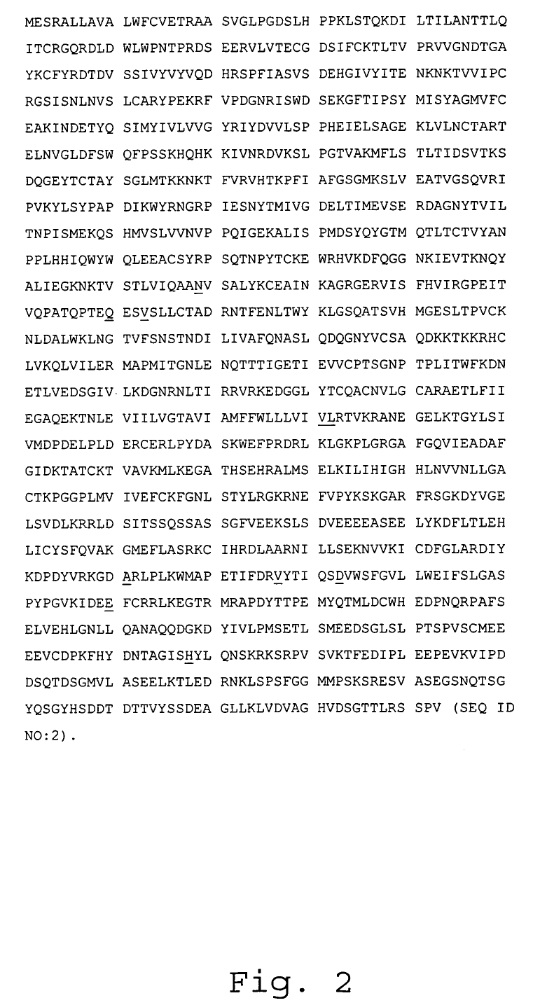

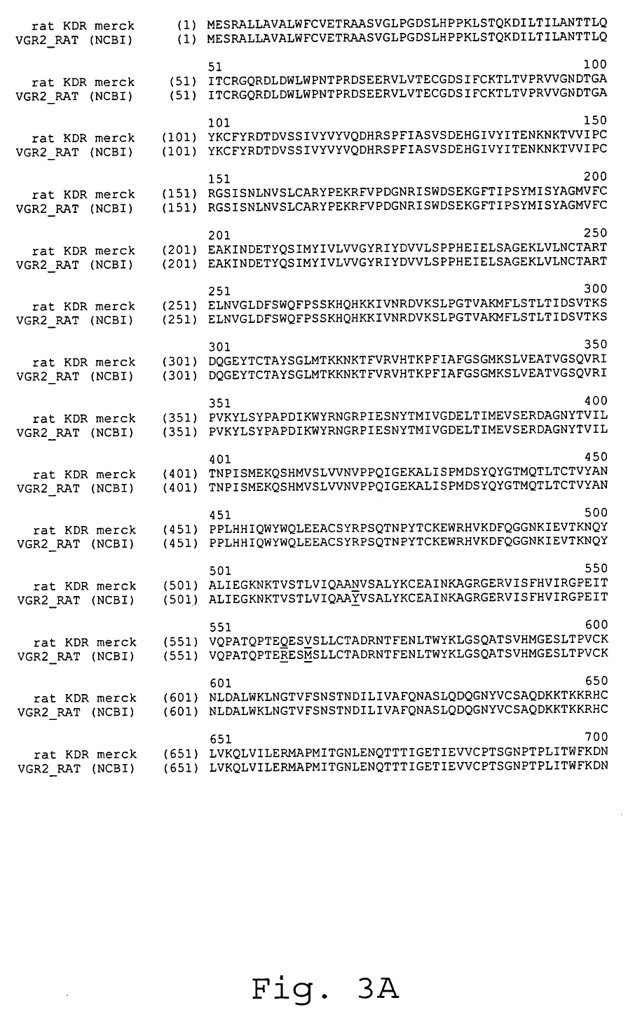

Results—An alignment of the published rat KDR amino acid sequence and the optimized rat KDR of the present invention is shown in FIG. 3A and FIG. 3B. The cDNA sequence of the optimized rat KDR is shown in FIGS. 1A-D. The deduced amino acid sequence of rat KDR is shown in FIG. 2. The optimized rat KDR of the present differs from the published rat KDR by ten amino acids as summarized in Table 1 below:

TABLE 1

| Residue in published | Corresponding residue in |
|---|---|
| rat KDR | optimized rat KDR |
| Tyr-519 | Asn |
| Arg-560 | Gln |
| Met-563 | Val |
| Val-753 | Ala |
| Leu-781 | Val |
| Val-782 | Leu |
| Pro-1061 | Ala |
| Ile-1077 | Val |
| Gly-1083 | Asp |
| Lys-1110 | Glu |

### EXAMPLE 2

### RT-PCR Cloning of the Intracellular Domain Coding Sequence of Rat KDR, RK7

Materials—Rat (Rattus norvegicus) lung poly A+RNA was purchased from Clontech. The PCR primers used are as follows:

| | | |
|---|---|---|
| rKDR-CD-S-NcoI | 5′-TTACCATGGAAGCGGGCCAATGAAGGGGAACTGAA-3′; | (SEQ ID NO:11) |
| rKDR-CD-A-KpnI | 5′-CCGGTACCAAATGAAAATCAAATGCGGCTACTTC-3′. | (SEQ ID NO:12) |

Methods—Rat KDR cytosolic domain was cloned from rat lung poly A+RNA by RT-PCR using Prostar Ultra HF RT-PCR System (Stratagene). The first strand cDNA, synthesized by reverse transcription primed with oligo(dT)₁₈, was subjected to high fidelity PCR using Pfu Turbo DNA polymerase and the aforementioned primers. A PCR product approximately 1.8 Kb in length was gel-purified, blunt-end ligated into SrfI site of PCRscript-Amp vector, and used to transform XL-10 Gold ultra-competent cells. Resulting ampicillin-resistant colonies were screened by PCR using the aforementioned primer pair and REDTaq ReadyMix PCR reaction mix (Sigma). Four colonies that yielded a 1.8 Kb PCR product were selected. Plasmid DNA derived from these colonies was analyzed by restriction digestions and DNA sequencing.

Results—RK7, a fragment of the optimized rat KDR that represents the intracellular (cytosolic) domain of the tyrosine kinase receptor, differs from the published rat KDR by four amino acids as summarized in Table 2 below:

TABLE 2

| Residue in published | |
|---|---|
| rat KDR sequence^{a} | Corresponding residue in RX7 |
| Pro-1061 (1065) | Ala |
| Ile-1077 (1081) | Val |
| Gly-1083 (1087) | Asp |
| Lys-1110 (1114) | Glu |

^{a}The number in parenthesis is the corresponding residue number in human KDR.

### EXAMPLE 3

### Site Directed Mutagenesis of Rat KDR Clone RK7

Materials—PCR reagents were purchased from Clontech. The following complementary mutagenic primers used are as follows:

| | | |
|---|---|---|
| Sense strand | 5′-AGTATACACAATTCAGAGTGGCGTGTGGTCTTTTGGTGTTTTG-3′; | (SEQ ID NO:13) |
| Anti-sense strand | 5′-CAAAACACCAAAAGACCACACGCCACTCTGAATTGTGTATAC-3′. | (SEQ ID NO:14) |

After synthesis, the PCR primers were PAGE-purified by Life Technologies, Inc. The underlined bases in the primers were to change the codon GAC (Asp) to GGC (Gly).

Methods—Asp-1083 of rat KDR clone RK7 was changed to Gly using QuickChange™ site-directed mutagenesis kit (Stratagene) modified by Clontech reagents. A 50 μl PCR reaction was set up by mixing 5.0 μl Advantage HF buffer (Clontech), 5.0 μl G-C melt (Clontech), 1.0 μl dNTP mix (Stratagene), 1.0 μl Advantage HF polymerase (Clontech), 125 ng of each primer, 50 ng of RK7 DNA and pure water. The reaction was conducted in a PTC-200 Peltier Thermal Cycler (MJ Research) with the following parameters: 95° C. for 30 s followed by 16 cycles each with 95° C. 30 s, 60° C. 1 min, 70° C. 10 min. The PCR product was digested with DpnI to destroy the wild type strands, and then, used to transform E. coli XL1-Blue super-competent cells. Plasmids prepared from the resulting colonies were sequenced to verify the presence of the desired mutation.

### EXAMPLE 4

### Comparison of Optimized Rat KDR to the Molecular Model of Human KDR

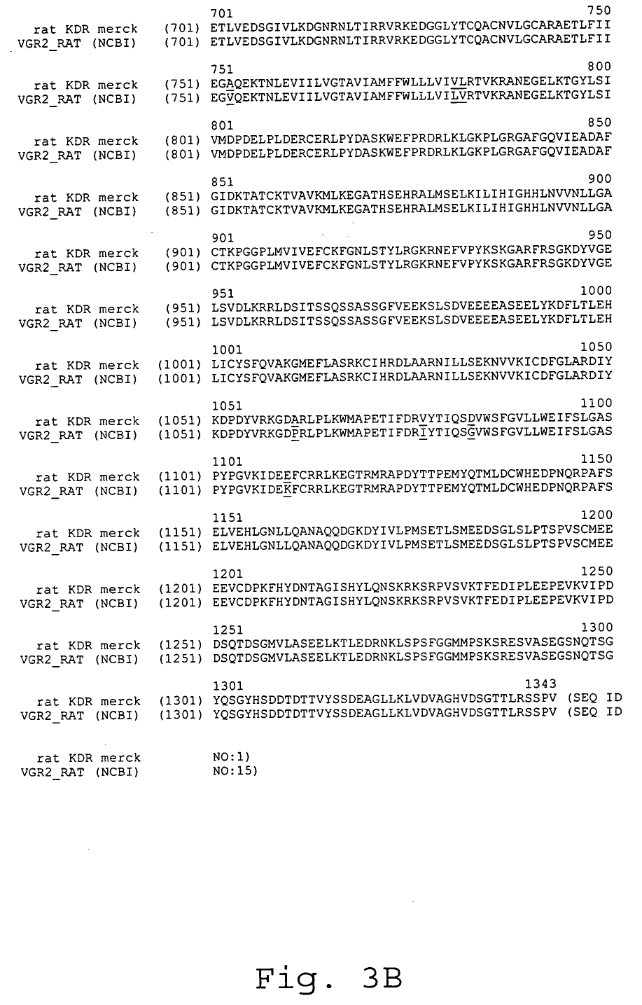

The optimized rat KDR of the present invention differs from the published rat KDR by ten amino acids. Four of these amino acid differences are located within the intracellular kinase domain of the protein: Asp at position 1083, Ala at position 1061, Val at position 1077 and Glu at position 1110. These four differences correspond to residues in the carboxyl-terminal of KDR and are conserved between the optimized rat KDR and human KDR (see Table 2 in Example 2). The published crystal structure of human KDR (McTigue et al., 1999, Structure 7:319-330) was used to explore the consequences of the sequence differences between the optimized rat KDR and the published rat KDR. FIG. 4 shows the location of the amino acids within the crystal structure of human KDR that correspond to the four amino acid differences noted between the optimized rat KDR and the published rat KDR sequence. In the human sequence, the four amino acids of interest are Ala (A) at 1065, Val (V) at 1081, Asp (D) at 1087, and Glu (E) at 1114.

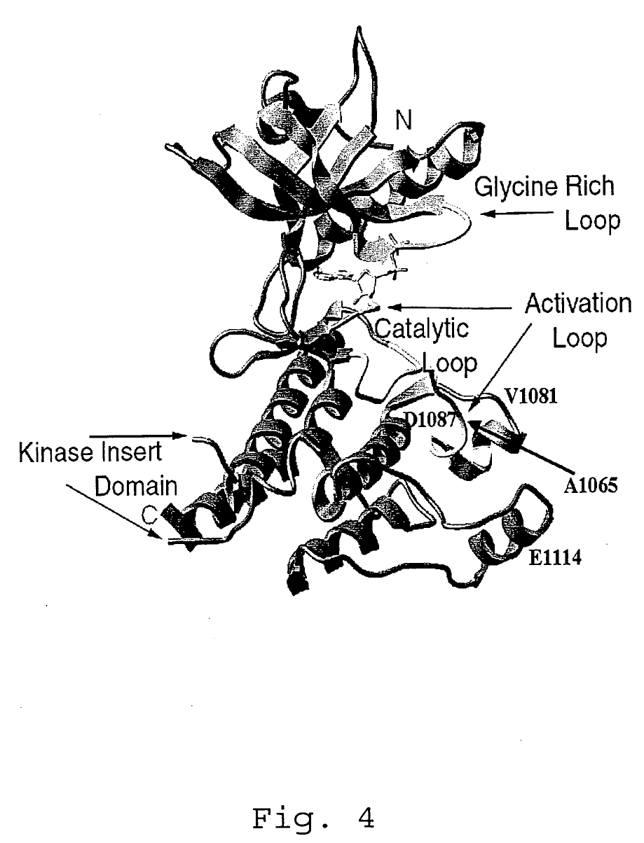

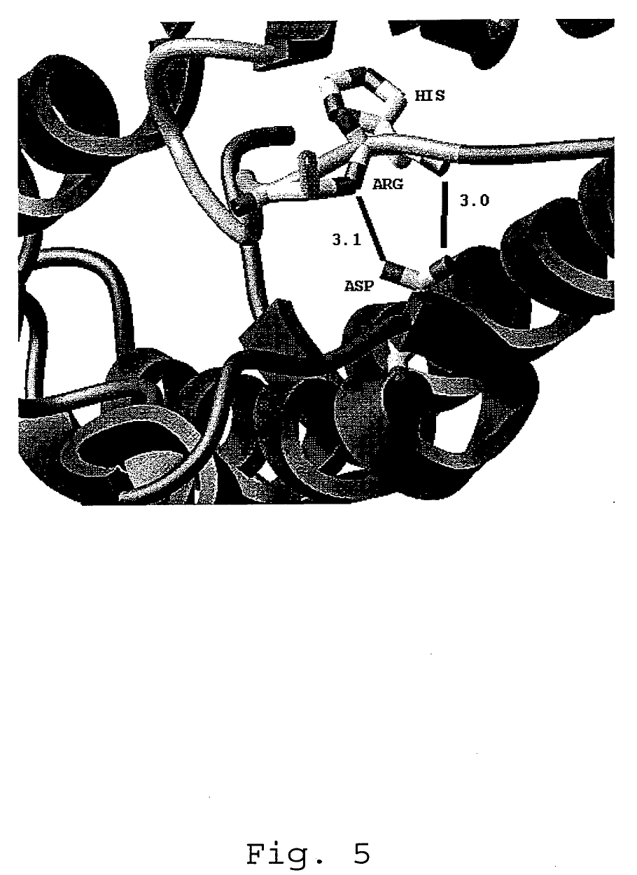

The amino acid difference at position 1083 (replacing Gly with Asp) of the rat KDR sequence generates the most notable difference between the optimized and published sequences. In human KDR, the corresponding Asp residue is located at position 1087 (see Table 2 in Example 2) on the alpha helix F (αF). Asp-1087 of human KDR is structurally close to the catalytic loop that mediates phosphotransfer. Asp-1087 is also hydrogen bonded to two backbone amide protons in the catalytic loop: His-1026 and Arg-1027 (see FIG. 5). This corresponding Asp residue at position 1083 of the optimized rat KDR is replaced with Gly in the published rat KDR sequence. With this alteration, the aforementioned hydrogen bonds would be eliminated as the side-chain of glycine does not contain any hydrogen bonding functionality. Since the catalytic loop is instrumental in the structure/function of kinases, and Asp-1087 is conversed in known tyrosine kinases, it is likely that the published rat KDR sequence would destabilize the catalytic loop and compromise the catalytic activity.

The remaining amino acid differences in the intracellular kinase domain may also affect the activity of rat KDR. In human KDR, Ala-1065 is located structurally close to the activation loop of the protein. Replacing Ala with a Pro at this position is likely to reduce the flexibility of the activation loop, which is required for kinase activity. The published rat KDR sequence indeed contains a Pro at position 1061, the position in rat KDR that corresponds to human residue number 1065, while the optimized rat KDR of the present invention has an Ala in that position. Additionally, although the remaining differences in the intracellular domain (Val-1077 to Ile, and Glu-1110 to Lys) are surface exposed, they could also have structural effects.

### EXAMPLE 5

### Expression of Recombinant Rat KDR Intracellular Domain, RK7, Tagged with GST-6×His

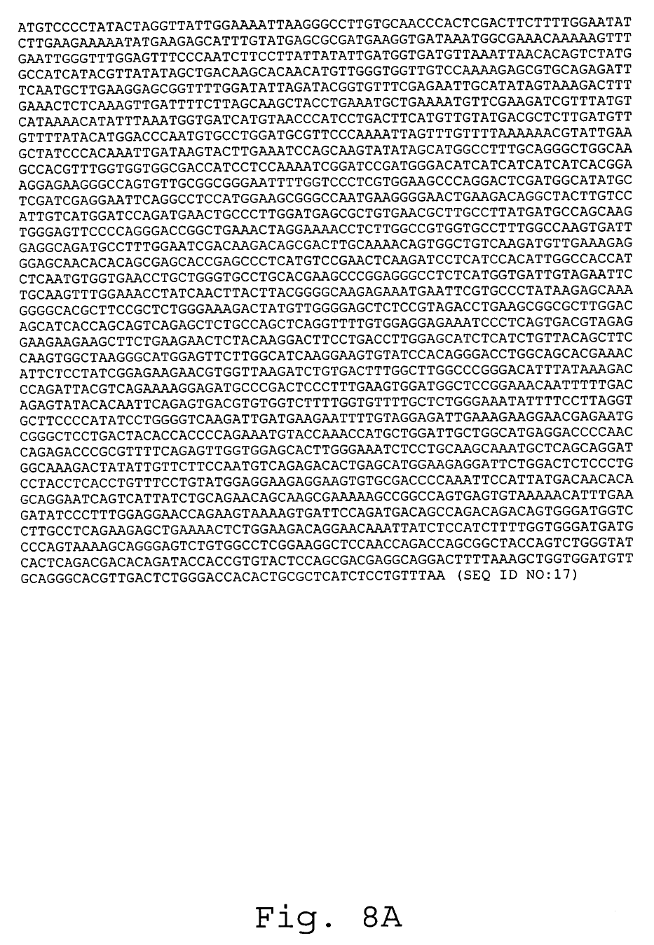

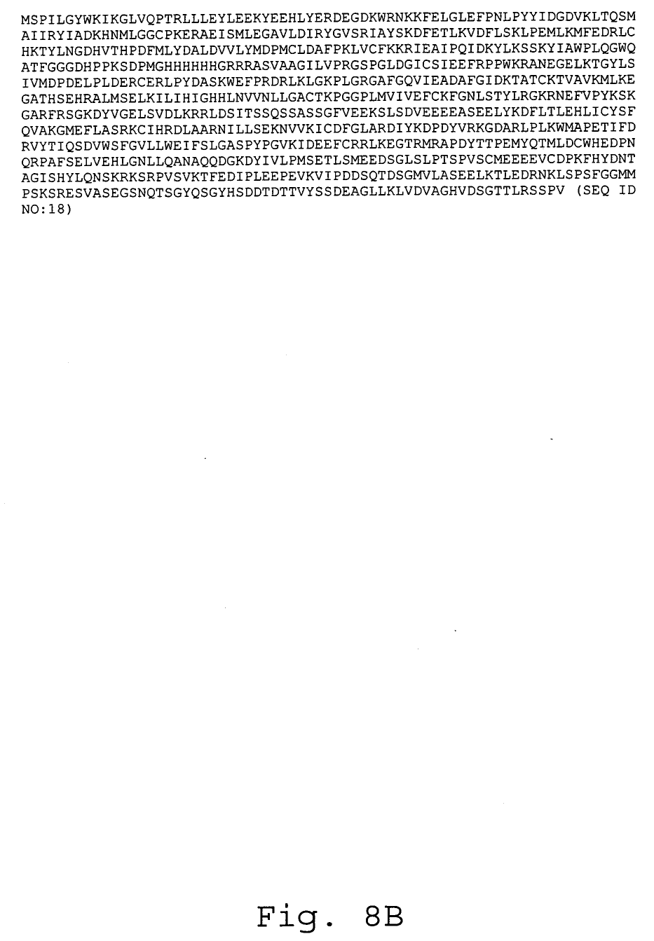

Recombinant baculovirus encoding rat KDR intracellular domain, RK7 (SEQ ID NO:16), was generated using a baculovirus expression kit (Pharmingen) according to a protocol recommended by the manufacturer. The resulting GST fusion protein, GST-RK7, is encoded by the nucleic acid sequence as set forth in SEQ ID NO:17 and has the amino acid sequence as set forth in SEQ ID NO:18 (see also FIGS. 8A and 8B). The KDR sequence in clone RK7 (in pPCRscript) was subcloned, using NcoI and KpnI, into pAcGHLT-A transfer vector down stream from and in-frame with the GST-6×His tag. The resulting transfer construct and BaculoGold baculovirus DNA were used to co-transfect insect cells (Sf9) seeded in a 60 mm dish. The culture medium (Po virus stock) of the Sf9 cells was collected 5 days after co-transfection. To confirm the expression of the GST-KDR fusion, an aliquot of the Po virus stock was used to infect 6×10⁵ healthy Sf9 cells. On the 6th day post-infection, the cells were lysed in 1.5 ml of a buffer containing 1% Triton X-100 and a protease inhibitor cocktail. GST-tagged protein(s) was precipitated from the lysate using glutathione-agarose beads. The beads were boiled in a Tris-glycine SDS sample buffer to release the bound proteins, which were then fractionated on a 8% polyacrylamide gel and subjected to Western blot analysis using a rabbit anti-KDR antibody (SC305, Santa Cruz Biotechnology). After the expression of GST-RK7 was confirmed by Western blot, an aliquot of the remaining Po virus stock was provided to Kemp Biotechnologies, Inc. (Frederick, Md.), which performed the subsequent steps of the expression. These included production of high titer recombinant baculovirus stocks, small scale expression runs aimed at optimizing the expression conditions and scaled-up expression of rat GST-RK7 fusion using a 10 liter bio-reactor.

### EXAMPLE 6

### Protein Purification of Wild Type and Mutant Rat KDR Fusions

The wild type and mutant rat KDR fusions were purified from Sf9 cell lysates by affinity chromatography using an AKTA Explorer chromatography system (AmershamPharmacia). About 30 gram of frozen Sf9 cell pellets were lysed in 4 volumes of lysis buffer containing 0.5% NP40, 1% Triton X-100, 135 mM NaCl, 1.5 mM H₂NaPO₄, 4.3 mM HNa₂PO₄, and COMPLETE™ protease inhibitor cocktail (Roche). The lysate was centrifuged at 40,000 RPM for 20 min in a Beckman ultracentrifuge using a type 45 Ti rotor. The supernatant was loaded onto a 5-ml GSTrap column (AmershamPharmacia) pre-equilibrated with the lysis buffer. The column was washed exhaustively with the lysis buffer, and subsequently, with phosphate-buffered saline (PBS) containing protease inhibitors. Bound proteins were then eluted with 10 mM glutathione in 50 mM Tris-HCl (pH 8.0). The eluted protein fractions were pooled, buffer-exchanged into Ni-NTA Binding Buffer (50 mM NaH₂PO₄, 300 mM NaCl, 10 mM imidazole, pH 8.0) using a Sephadex G-25 desalting column, and loaded onto a Ni-NTA Superflow (Qiagen) column (bed volume: 5 ml) pre-equilibrated with the same buffer. The Ni-NTA column was washed exhaustively with Ni-NTA Binding Buffer followed by Ni-NTA Wash Buffer (50 mM NaH₂PO₄, 300 mM NaCl, 20 mM imidazole, pH=8.0). The bound protein(s) was eluted with Ni-NTA Elution Buffer (50 mM NaH₂PO₄, 300 mM NaCl, 250 mM imidazole, pH 8.0). The eluted protein fractions were pooled and dialyzed against 50% glycerol, 2 mM DTT, 50 mM Tris-HCl (pH 7.4) and stored in small aliquots at −20° C. The protein concentrations of the dialyzed fusion proteins were determined using Coomassie Plus Protein Assay (Pierce) with BSA as standard.

### EXAMPLE 7

### Autophosphorylation Assay of Rat KDR

To determine the functional consequence of the substitution of an Asp residue at position 1083 of optimized rat KDR with Gly, as occurs in the published rat KDR sequence, RK7 (the fragment representing the intracellular domain of optimized rat KDR) was altered at position 1083 to contain a Gly (G) by site-directed mutagenesis. Both RK7 and the RK7(G1083) variant were expressed as GST-tagged fusion proteins in insect cells using a baculovirus system. The proteins were evaluated in terms of their abilities to autophosphorylate.

Purified recombinant GST-tagged RK7 (2.5 μg/ml) and RK7 (G1083) (2.5 μg/ml) were pre-incubated separately at 25° C. for 10 min in 10 mM MgCl₂, 2 mM MnCl₂, 100 mM NaCl, 50 mM Tris-HCl (pH 7.2), 0.5 mg/ml BSA, 0.5 mM Na₃VO₄ and 0.5 mM TCEP (Tris[2-carboxyethylphosphine] hydrochloride (Pierce)) in two microcentrifuge tubes. The autophosphorylation reactions were initiated by addition of a small volume of 10 mM ATP to each of the tubes to yield a final ATP concentration of 1 mM. Aliquots were withdrawn from each of the reactions at various times and mixed immediately with an equal volume of 50 mM EDTA to stop the autophosphorylation reaction. The EDTA-containing samples were then mixed with 2× Tris-Glycine SDS sample buffer (Novex) containing 100 mM dithiothreitol and boiled for 5 min. The samples were electrophoresed on two 8% acrylamide-Tris-Glycine gels (Novex). The proteins separated on the gels were then transferred to two PVDF membranes (Immobilon™-P, Millipore) using Xcell II Blot Module (Novex). One membrane was probed with a mouse monoclonal anti-PY antibody (4G10, Upstate Biotechnologies, Inc), and the second membrane was probed with a mouse monoclonal anti-KDR antibody (SC-625 1, Santa Cruz Biotechnology). The membranes were developed using a sheep anti-mouse antibody conjugated to horseradish peroxidase and ECL (AmershamPharmacia).

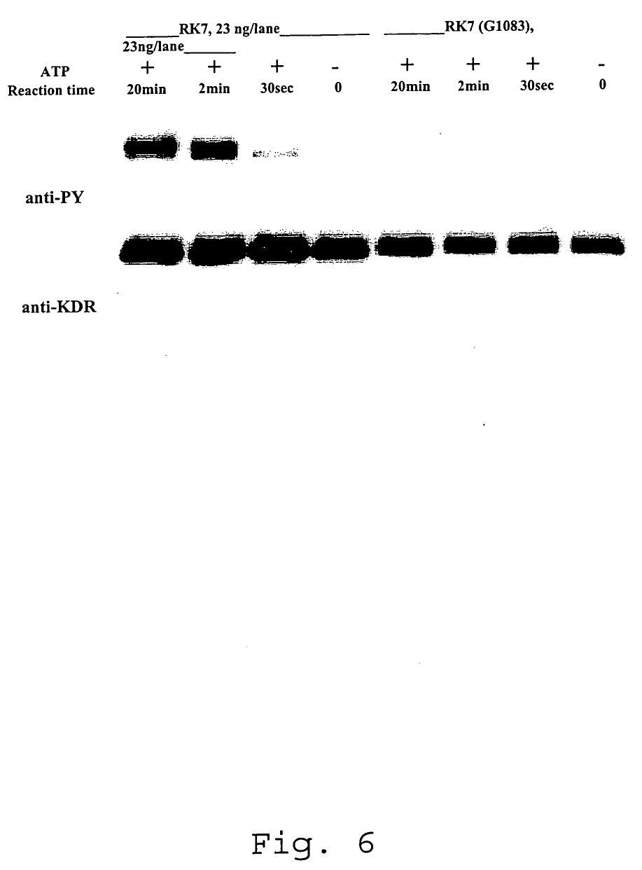

The recombinant RK7 protein exhibited rapid autophosphorylation when incubated with ATP, while RK7 (G1083) showed no detectable autophosphorylation activity (FIG. 6). This indicates that the presence of a Gly at position 1083 causes a complete loss of kinase activity of the rat KDR intracellular domain. Therefore the published rat KDR sequence, containing a Gly at position 1083, appears to represent an inactive kinase.

### EXAMPLE 8

### Tyrosine Phosphorylation of Rat KDR

To further investigate the functional consequence of substitution of the Asp residue at position 1083 of the optimized rat KDR with Gly, as occurs in the published rat KDR sequence, purified GST-RK7 and RK7 (G1083) were evaluated in terms of their abilities to phosphorylate a synthetic biotinylated peptide substrate.

A master reaction mix was prepared which contained 1 μM substrate (biotinylated EQEDEPEGDYFEWLE; SEQ ID NO:10), 10 μM ATP, 10 mM MgCl₂, 2 mM MnCl₂, 100 mM NaCl, 50 mM Tris-HCl (pH 7.2), 0.5 mg/ml BSA, 0.5 mM Na₃VO₄, and 0.5 mM TCEP. The master mix was distributed to the wells (50 μl per well) of a black 96-well plate. The kinase reactions were initiated by addition of 10 μl of GST-RK7 or GST-RK7 (G1083) pre-serial-diluted in the above buffer less the substrate and ATP. Each reaction was allowed to proceed for 35 min at room temperature with shaking and then stopped by addition of 50 μl of a quench buffer containing 0.8 μg/ml Eu(K)-PT-66 (an europium cryptate-labeled anti-phosphotyrosine antibody), 10 μg/ml streptavidin-XL665, 100 mM EDTA, 0.5 mM KF, 0.1% Triton X-100. The quenched reactions were incubated for 5 hours at room temperature and then read in Discovery (Packard), a time-resolved fluorescence detector.

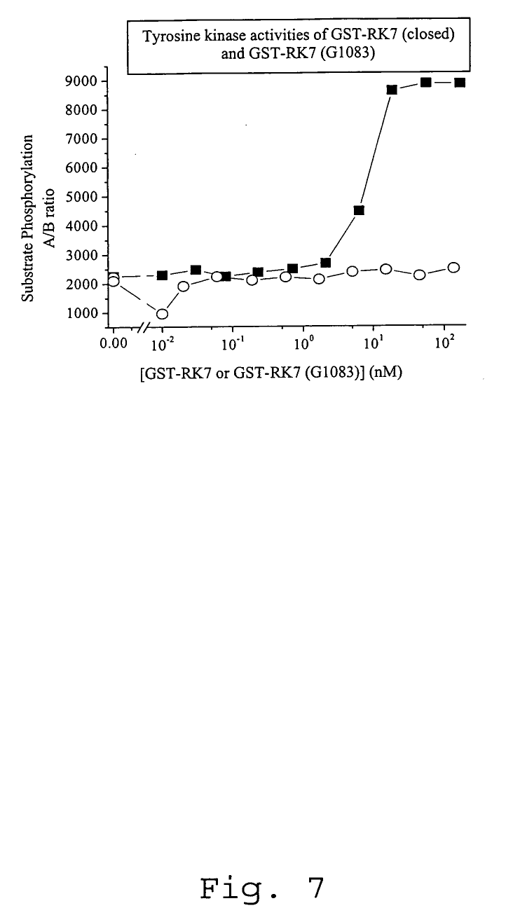

The recombinant RK7 was able to tyrosine phosphorylate the synthetic peptide, while RK7 (G1083) showed no detectable tyrosine kinase activity in this assay (FIG. 7). Again, this data indicates that the presence of Gly at position 1083 of the published rat KDR sequence causes a complete loss of the kinase activity of the rat KDR intracellular domain. Thus, the published rat KDR sequence appears to represent an inactive kinase.

1. An isolated nucleic acid molecule encoding a rat KDR protein, wherein said nucleic acid molecule comprises a nucleotide sequence encoding a rat KDR protein retaining an aspartic acid residue at position 1083.

2-4. (canceled)

5. An isolated nucleic acid molecule encoding a rat KDR protein comprising a nucleotide sequence encoding the amino acid sequence as set forth in SEQ ID NO:2.

6. An expression vector for expressing a rat KDR protein in a recombinant cell wherein said expression vector comprises a nucleic acid molecule of claim 5.

7. A host cell which expresses a recombinant rat KDR protein wherein said host cell contains the expression vector of claim 6.

8. A process of expressing a rat KDR protein in a recombinant host cell, comprising:
(a) transfecting the expression vector of claim 6 into a suitable host cell; and
(b) culturing the host cells of step (a) under conditions which allow expression of said rat KDR protein from said expression vector.

9. An isolated nucleic acid molecule encoding a rat KDR protein consisting of the DNA molecule as set forth in SEQ ID NO:1.

10. An expression vector for expressing a rat KDR protein in a recombinant cell wherein said expression vector comprises a nucleic acid molecule of claim 9.

11. A host cell which expresses a recombinant rat KDR protein wherein said host cell contains the expression vector of claim 10.

12. (canceled)

13. An isolated nucleic acid molecule encoding an intracellular portion of a rat KDR protein, wherein said nucleic acid molecule comprises a nucleotide sequence encoding from about amino acid 783 to about amino acid 1343 as set forth in SEQ ID NO:2, wherein position 1083 is an aspartic acid residue.

14. An expression vector for expressing an intracellular portion of a rat KDR protein in a recombinant cell wherein said expression vector comprises a nucleic acid molecule of claim 13.

15. A host cell which expresses a recombinant intracellular portion of a rat KDR protein wherein said host cell contains the expression vector of claim 14.

16. (canceled)

17. An isolated nucleic acid molecule encoding a soluble KDR fusion protein wherein said nucleic acid molecule comprises a nucleotide sequence encoding from about amino acid 783 to about amino acid 1343 of rat KDR as set forth in SEQ ID NO:2, wherein position 1083 is an aspartic acid residue.

18. An expression vector for expressing a soluble KDR fusion protein in a recombinant cell wherein said expression vector comprises a nucleic acid molecule of claim 17.

19-20. (canceled)

21. An isolated nucleic acid molecule of claim 17 which encodes GST-RK7, as set forth in SEQ ID NO:17.

22-27. (canceled)

28. A purified rat KDR protein which comprises the amino acid sequence as set forth in SEQ ID NO:2.

29. A purified rat KDR protein of claim 28 which is a product of a DNA expression vector contained within a recombinant host cell.

30. A substantially pure membrane preparation comprising the rat KDR protein purified from the recombinant host cell of claim 29.

31. A purified protein fragment which is an intracellular portion of a rat KDR protein, comprising from about amino acid 783 to about amino acid 1343 as set forth in SEQ ID NO:2, wherein position 1083 is an aspartic acid residue.

32-33. (canceled)

34. A purified KDR fusion protein which is characterized by an intracellular portion of a rat KDR protein, comprising from about amino acid 783 to about amino acid 1343 as set forth in SEQ ID NO:2, wherein position 1083 is an aspartic acid residue.

35-36. (canceled)

37. The purified KDR fusion protein of claim 34 which is GST-RK7, as set forth in SEQ ID NO:18.

38-41. (canceled)

42. A method of selecting a compound which modulates rat KDR which comprises a biological assay wherein a test compound is added in combination with a rat KDR protein or protein fragment and a substrate, said substrate being involved in a measurable interaction at a domain of interest with wild-type rat KDR such that said test compound interacts with said rat KDR protein or protein fragment, resulting in a measurable change in KDR:substrate activity.

43. A method of claim 42, wherein said test compound is an compound antagonist, resulting in a measurable decrease in KDR:substrate activity.

44. A method of claim 42, wherein said test compound is an compound agonist, resulting in a measurable increase in KDR:substrate activity.


SEQUENCE LISTING (18 sequences)

SEQ ID NO: 1 - DNA, 5693 bp, Rattus norvegicus
  CDS (38)...(4069)
gaccgagaaagcatctgtgcccagcgcgaggtgcaggatggagagcagggcgctgctagctgtcgctctgtggttctgcgtggagacccgagccgcctctgtgggtttgcctggcgattccctccatccacccaagctcagcacacaaaaagacatacttacaattttggcaaatacaacccttcagattacttgcaggggacagagggacctggattggctttggcccaacactccgcgtgactctgaggaaagggtgttggtgactgagtgtggcgacagtatcttctgcaagacactcacagttcccagagtggttggaaatgatactggagcctacaagtgcttctatcgggacaccgatgtctcctccatcgtttatgtctatgttcaagatcacaggtcaccattcatcgcctctgtcagtgacgagcatggcatcgtgtacatcactgagaacaagaacaaaactgtggtgatcccatgccgagggtcgatttcaaacctcaacgtgtcactttgtgctaggtatccagaaaagagatttgttccggatggaaacagaatttcctgggacagcgagaaaggctttactatccccagttacatgatcagctatgccggcatggtcttctgtgaggcaaagattaatgatgaaacgtatcagtctatcatgtacatagttctggttgtaggatataggatttatgatgtggtcctgagcccccctcatgaaattgagctatctgccggagaaaagcttgtcttaaattgtacagcaagaacagagctcaacgtggggcttgatttcagctggcaattcccgtcctcaaagcatcagcataagaagattgtaaaccgggatgtgaaatcccttcctgggactgtggcaaagatgtttttgagcaccttgaccatagacagtgtgaccaagagtgaccaaggagaatacacctgcacagcgtacagtggactgatgaccaagaaaaataaaacatttgtccgagttcatacaaaaccttttattgcttttggtagcgggatgaaatctttggtggaagccactgtgggcagccaagtccgaatccctgtgaagtatctcagttacccagctcctgatatcaaatggtacagaaatggacgacccattgagtccaattacacaatgatcgttggtgatgaactcaccatcatggaagtgagtgaaagagatgcgggaaactacacggtcatcctcaccaatcccatttcaatggagaaacagagccacatggtctctctggttgtgaatgttccaccccagatcggtgagaaagccttgatctctcctatggattcctaccagtatggcaccatgcagacgctgacatgcacagtctatgccaaccctcccctgcaccacatccaatggtactggcagctagaagaagcatgctcctacaggcccagccaaacaaacccatatacttgtaaagaatggagacacgtgaaggatttccaggggggaaataagatcgaagtcaccaaaaaccaatatgccctaattgaaggaaaaaacaaaactgtaagtactctggtcatccaggctgccaacgtgtccgcattatacaaatgtgaagccatcaacaaagcaggacgaggagagagggtcatctccttccatgtgatcaggggtcctgaaattactgtccagcctgctacccagccaaccgagcaggagagtgtgtctctattgtgcactgcagatagaaacacgtttgagaacctcacgtggtacaagcttggctcacaggcaacatcggtccacatgggcgaatcactcacaccagtttgcaagaacttggacgctctttggaaactgaatggcaccgtgttttctaacagcacaaacgacatcttgattgtggcattccagaatgcctccctgcaggaccaaggcaactatgtctgctctgctcaagacaagaagaccaagaaaagacattgcctagtcaagcagctcgtcatcctagagcgcatggcacccatgatcactggaaatctggagaatcagacaacaaccattggtgagaccatcgaagttgtttgtccaacatctggaaaccctacccccctcattacatggttcaaagacaatgagacccttgtagaagattcaggcattgtactaaaagacgggaaccggaacctaactatccgaagggtgaggaaggaagacgggggcctctacacctgccaggcctgcaatgtccttggctgtgcaagagcagagacactcttcataatagaaggtgcccaggaaaagaccaacttggaagtcattattctcgtcggcactgcagtgatcgccatgttcttctggctacttcttgtcattgttctacggaccgttaagcgggccaatgaaggggaactgaagacaggctacttgtccattgtcatggatccagatgaactgcccttggatgagcgctgtgaacgcttgccttatgatgccagcaagtgggagttccccagggaccggctgaaactaggaaaacctcttggccgtggtgcctttggccaagtgattgaggcagatgcctttggaatcgacaagacagcgacttgcaaaacagtggctgtcaagatgttgaaagagggagcaacacacagcgagcaccgagccctcatgtccgaactcaagatcctcatccacattggccaccatctcaatgtggtgaacctgctgggtgcctgcacgaagcccggagggcctctcatggtgattgtagaattctgcaagtttggaaacctatcaacttacttacggggcaagagaaatgaattcgtgccctataagagcaaaggggcacgcttccgctctgggaaagactatgttggggagctctccgtagacctgaagcggcgcttggacagcatcaccagcagtcagagctctgccagctcaggttttgtggaggagaaatccctcagtgacgtagaggaagaagaagcttctgaagaactctacaaggacttcctgaccttggagcatctcatctgttacagcttccaagtggctaagggcatggagttcttggcatcaaggaagtgtatccacagggacctggcagcacgaaacattctcctatcggagaagaacgtggttaagatctgtgactttggcttggcccgggacatttataaagacccagattacgtcagaaaaggagatgcccgactccctttgaagtggatggctccggaaacaatttttgacagagtatacacaattcagagtgacgtgtggtcttttggtgttttgctctgggaaatattttccttaggtgcttccccatatcctggggtcaagattgatgaagaattttgtaggagattgaaagaaggaacgagaatgcgggctcctgactacaccaccccagaaatgtaccaaaccatgctggattgctggcatgaggaccccaaccagagacccgcgttttcagagttggtggagcacttgggaaatctcctgcaagcaaatgctcagcaggatggcaaagactatattgttcttccaatgtcagagacactgagcatggaagaggattctggactctccctgcctacctcacctgtttcctgtatggaggaagaggaagtgtgcgaccccaaattccattatgacaacacagcaggaatcagtcattatctgcagaacagcaagcgaaaaagccggccagtgagtgtaaaaacatttgaagatatccctttggaggaaccagaagtaaaagtgattccagatgacagccagacagacagtgggatggtccttgcctcagaagagctgaaaactctggaagacaggaacaaattatctccatcttttggtgggatgatgcccagtaaaagcagggagtctgtggcctcggaaggctccaaccagaccagcggctaccagtctgggtatcactcagacgacacagataccaccgtgtactccagcgacgaggcaggacttttaaagctggtggatgttgcagggcacgttgactctgggaccacactgcgctcatctcctgtttaaaaggaagtggccctgtcccgtccccgcccccaactcctggaaataactcgagaggtgctgcttagattttcaagtgttgttctttccaccactcggaagtagccgcatttgattttcatttcagaagagggacctcagacggcaagaagcttgtcctcagggcatttccagaaaaatgcccatgacccaagaatgtgttgactatactctcttttccattggtttaaaaatcctatatattgtgccctgctgcgggtctcactaccagttaaaacaaaagacgttcaaacagcggctctatcctccaagaagtagccatacccaggcaatggagccctctgtgaaactggataaaatgggcgatgttagtgctttgtgtgttgggatgggtgagatgtcccagggctgagtctacctaaaaggctttgtggaggatgtgggctatgagccaagtgttaagtgtgagatgtggactggtaggaaggaaggagcaagctcgctcagagagcggttggagcctgcagatgcattgtgctggctgtggtggaggtgagcatgtggcctgtcaggaaacgccaaggcggctgtcggggtttggttttggaaggttgcgtgctcttcacggttgggctacaggcgagttccctgtgctgtttcctactcctaatgagagttccttccggactcttacgtgtctcctggcctagccccaggaaggaaatgacgcagcttgctcctcatctcccaggctgtgccttaactcagaatactaaaagagagggactttggccgaggctccgctccttgtcatgctgaagaactgtgagaacacaacagaaactcagggtttctgctgggtggatacccacttgtctgccctggtggcagtgtctgagggttttgtcaagtggcgatggtaaaggctcagacaggatgtatccctttgttcttcctctaactccacttctgtcttgccacacccccccctccccagtgctcagtattttagctttgtggccacgtgatggcagaaggtcttaattggttggttttgctctccagataaaatcactagtcagatttcgaaattactttatagccaaggtctgataacatctactgtatcgtttagaatttaacatataaagctgtgtctactggttttttttttttttgcccttgggcatatgtttttcaaaagagaaactacttttcatttggtaccatagcgtgacgagcaggggccaatgactgtaaaacatgctgtggcacatatatttatagtctgttatgtggaacaaatgtaatatattgaaactttatattatatataaggaactttgtactatccgcatttcgtatcagtattatgtagcatgacagagactgtgaggtctgagcagctggtggctcaggacgttgagaaactcgaaggaatcctttcgtgaggatgcgcagctatccctacccatctctctcacctcaaacggaggagaaaggggaatcagagataatgtgagtgtgtccttgttctctgttcttaggaggaatgttcttaccaactgttcatacgctttataaaccaataaatgtattctgagtaaagaaaaaaaaaaaaaaaaaa

SEQ ID NO: 2 - protein, 1343 aa, Rattus norvegicus
MESRALLAVALWFCVETRAASVGLPGDSLHPPKLSTQKDILTILANTTLQITCRGQRDLDWLWPNTPRDSEERVLVTECGDSIFCKTLTVPRVVGNDTGAYKCFYRDTDVSSIVYVYVQDHRSPFIASVSDEHGIVYITENKNKTVVIPCRGSISNLNVSLCARYPEKRFVPDGNRISWDSEKGFTIPSYMISYAGMVFCEAKINDETYQSIMYIVLVVGYRIYDVVLSPPHEIELSAGEKLVLNCTARTELNVGLDFSWQFPSSKHQHKKIVNRDVKSLPGTVAKMFLSTLTIDSVTKSDQGEYTCTAYSGLMTKKNKTFVRVHTKPFIAFGSGMKSLVEATVGSQVRIPVKYLSYPAPDIKWYRNGRPIESNYTMIVGDELTIMEVSERDAGNYTVILTNPISMEKQSHMVSLVVNVPPQIGEKALISPMDSYQYGTMQTLTCTVYANPPLHHIQWYWQLEEACSYRPSQTNPYTCKEWRHVKDFQGGNKIEVTKNQYALIEGKNKTVSTLVIQAANVSALYKCEAINKAGRGERVISFHVIRGPEITVQPATQPTEQESVSLLCTADRNTFENLTWYKLGSQATSVHMGESLTPVCKNLDALWKLNGTVFSNSTNDILIVAFQNASLQDQGNYVCSAQDKKTKKRHCLVKQLVILERMAPMITGNLENQTTTIGETIEVVCPTSGNPTPLITWFKDNETLVEDSGIVLKDGNRNLTIRRVRKEDGGLYTCQACNVLGCARAETLFIIEGAQEKTNLEVIILVGTAVIAMFFWLLLVIVLRTVKRANEGELKTGYLSIVMDPDELPLDERCERLPYDASKWEFPRDRLKLGKPLGRGAFGQVIEADAFGIDKTATCKTVAVKMLKEGATHSEHRALMSELKILIHIGHHLNVVNLLGACTKPGGPLMVIVEFCKFGNLSTYLRGKRNEFVPYKSKGARFRSGKDYVGELSVDLKRRLDSITSSQSSASSGFVEEKSLSDVEEEEASEELYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHEDPNQRPAFSELVEHLGNLLQANAQQDGKDYIVLPMSETLSMEEDSGLSLPTSPVSCMEEEEVCDPKFHYDNTAGISHYLQNSKRKSRPVSVKTFEDIPLEEPEVKVIPDDSQTDSGMVLASEELKTLEDRNKLSPSFGGMMPSKSRESVASEGSNQTSGYQSGYHSDDTDTTVYSSDEAGLLKLVDVAGHVDSGTTLRSSPV

SEQ ID NO: 3 - DNA, 17 bp, Artificial Sequence
  oligonucleotide
tggttctgcgtggagac

SEQ ID NO: 4 - DNA, 18 bp, Artificial Sequence
  oligonucleotide
ttctccggcagatagctc

SEQ ID NO: 5 - DNA, 18 bp, Artificial Sequence
  oligonucleotide
gaactgcccttggatgag

SEQ ID NO: 6 - DNA, 18 bp, Artificial Sequence
  oligonucleotide
gcaggttcaccacattga

SEQ ID NO: 7 - DNA, 18 bp, Artificial Sequence
  oligonucleotide
ctgctagctgtcgctctg

SEQ ID NO: 8 - DNA, 18 bp, Artificial Sequence
  oligonucleotide
ctgcagtgattgccatgt

SEQ ID NO: 9 - DNA, 18 bp, Artificial Sequence
  oligonucleotide
gggcacgaattcatttct

SEQ ID NO: 10 - protein, 15 aa, Artificial Sequence
  peptide
EQEDEPEGDYFEWLE

SEQ ID NO: 11 - DNA, 35 bp, Artificial Sequence
  oligonucleotide
ttaccatggaagcgggccaatgaaggggaactgaa

SEQ ID NO: 12 - DNA, 34 bp, Artificial Sequence
  oligonucleotide
ccggtaccaaatgaaaatcaaatgcggctacttc

SEQ ID NO: 13 - DNA, 43 bp, Artificial Sequence
  oligonucleotide
agtatacacaattcagagtggcgtgtggtcttttggtgttttg

SEQ ID NO: 14 - DNA, 42 bp, Artificial Sequence
  oligonucleotide
caaaacaccaaaagaccacacgccactctgaattgtgtatac

SEQ ID NO: 15 - protein, 1343 aa, Rattus norvegicus
MESRALLAVALWFCVETRAASVGLPGDSLHPPKLSTQKDILTILANTTLQITCRGQRDLDWLWPNTPRDSEERVLVTECGDSIFCKTLTVPRVVGNDTGAYKCFYRDTDVSSIVYVYVQDHRSPFIASVSDEHGIVYITENKNKTVVIPCRGSISNLNVSLCARYPEKRFVPDGNRISWDSEKGFTIPSYMISYAGMVFCEAKINDETYQSIMYIVLVVGYRIYDVVLSPPHEIELSAGEKLVLNCTARTELNVGLDFSWQFPSSKHQHKKIVNRDVKSLPGTVAKMFLSTLTIDSVTKSDQGEYTCTAYSGLMTKKNKTFVRVHTKPFIAFGSGMKSLVEATVGSQVRIPVKYLSYPAPDIKWYRNGRPIESNYTMIVGDELTIMEVSERDAGNYTVILTNPISMEKQSHMVSLVVNVPPQIGEKALISPMDSYQYGTMQTLTCTVYANPPLHHIQWYWQLEEACSYRPSQTNPYTCKEWRHVKDFQGGNKIEVTKNQYALIEGKNKTVSTLVIQAAYVSALYKCEAINKAGRGERVISFHVIRGPEITVQPATQPTERESMSLLCTADRNTFENLTWYKLGSQATSVHMGESLTPVCKNLDALWKLNGTVFSNSTNDILIVAFQNASLQDQGNYVCSAQDKKTKKRHCLVKQLVILERMAPMITGNLENQTTTIGETIEVVCPTSGNPTPLITWFKDNETLVEDSGIVLKDGNRNLTIRRVRKEDGGLYTCQACNVLGCARAETLFIIEGVQEKTNLEVIILVGTAVIAMFFWLLLVILVRTVKRANEGELKTGYLSIVMDPDELPLDERCERLPYDASKWEFPRDRLKLGKPLGRGAFGQVIEADAFGIDKTATCKTVAVKMLKEGATHSEHRALMSELKILIHIGHHLNVVNLLGACTKPGGPLMVIVEFCKFGNLSTYLRGKRNEFVPYKSKGARFRSGKDYVGELSVDLKRRLDSITSSQSSASSGFVEEKSLSDVEEEEASEELYKDFLTLEHLICYSFQVAKGMEFLASRKCIHRDLAARNILLSEKNVVKICDFGLARDIYKDPDYVRKGDPRLPLKWMAPETIFDRIYTIQSGVWSFGVLLWEIFSLGASPYPGVKIDEKFCRRLKEGTRMRAPDYTTPEMYQTMLDCWHEDPNQRPAFSELVEHLGNLLQANAQQDGKDYIVLPMSETLSMEEDSGLSLPTSPVSCMEEEEVCDPKFHYDNTAGISHYLQNSKRKSRPVSVKTFEDIPLEEPEVKVIPDDSQTDSGMVLASEELKTLEDRNKLSPSFGGMMPSKSRESVASEGSNQTSGYQSGYHSDDTDTTVYSSDEAGLLKLVDVAGHVDSGTTLRSSPV

SEQ ID NO: 16 - protein, 558 aa, Rattus norvegicus
KRANEGELKTGYLSIVMDPDELPLDERCERLPYDASKWEFPRDRLKLGKPLGRGAFGQVIEADAFGIDKTATCKTVAVKMLKEGATHSEHRALMSELKILIHIGHHLNVVNLLGACTKPGGPLMVIVEFCKFGNLSTYLRGKRNEFVPYKSKGARFRSGKDYVGELSVDLKRRLDSITSSQSSASSGFVEEKSLSDVEEEEASEELYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHEDPNQRPAFSELVEHLGNLLQANAQQDGKDYIVLPMSETLSMEEDSGLSLPTSPVSCMEEEEVCDPKFHYDNTAGISHYLQNSKRKSRPVSVKTFEDIPLEEPEVKVIPDDSQTDSGMVLASEELKTLEDRNKLSPSFGGMMPSKSRESVASEGSNQTSGYQSGYHSDDTDTTVYSSDEAGLLKLVDVAGHVDSGTTLRSSPV

SEQ ID NO: 17 - DNA, 2463 bp, Rat GST fusion
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatccgatgggacatcatcatcatcatcacggaaggagaagggccagtgttgcggcgggaattttggtccctcgtggaagcccaggactcgatggcatatgctcgatcgaggaattcaggcctccatggaagcgggccaatgaaggggaactgaagacaggctacttgtccattgtcatggatccagatgaactgcccttggatgagcgctgtgaacgcttgccttatgatgccagcaagtgggagttccccagggaccggctgaaactaggaaaacctcttggccgtggtgcctttggccaagtgattgaggcagatgcctttggaatcgacaagacagcgacttgcaaaacagtggctgtcaagatgttgaaagagggagcaacacacagcgagcaccgagccctcatgtccgaactcaagatcctcatccacattggccaccatctcaatgtggtgaacctgctgggtgcctgcacgaagcccggagggcctctcatggtgattgtagaattctgcaagtttggaaacctatcaacttacttacggggcaagagaaatgaattcgtgccctataagagcaaaggggcacgcttccgctctgggaaagactatgttggggagctctccgtagacctgaagcggcgcttggacagcatcaccagcagtcagagctctgccagctcaggttttgtggaggagaaatccctcagtgacgtagaggaagaagaagcttctgaagaactctacaaggacttcctgaccttggagcatctcatctgttacagcttccaagtggctaagggcatggagttcttggcatcaaggaagtgtatccacagggacctggcagcacgaaacattctcctatcggagaagaacgtggttaagatctgtgactttggcttggcccgggacatttataaagacccagattacgtcagaaaaggagatgcccgactccctttgaagtggatggctccggaaacaatttttgacagagtatacacaattcagagtgacgtgtggtcttttggtgttttgctctgggaaatattttccttaggtgcttccccatatcctggggtcaagattgatgaagaattttgtaggagattgaaagaaggaacgagaatgcgggctcctgactacaccaccccagaaatgtaccaaaccatgctggattgctggcatgaggaccccaaccagagacccgcgttttcagagttggtggagcacttgggaaatctcctgcaagcaaatgctcagcaggatggcaaagactatattgttcttccaatgtcagagacactgagcatggaagaggattctggactctccctgcctacctcacctgtttcctgtatggaggaagaggaagtgtgcgaccccaaattccattatgacaacacagcaggaatcagtcattatctgcagaacagcaagcgaaaaagccggccagtgagtgtaaaaacatttgaagatatccctttggaggaaccagaagtaaaagtgattccagatgacagccagacagacagtgggatggtccttgcctcagaagagctgaaaactctggaagacaggaacaaattatctccatcttttggtgggatgatgcccagtaaaagcagggagtctgtggcctcggaaggctccaaccagaccagcggctaccagtctgggtatcactcagacgacacagataccaccgtgtactccagcgacgaggcaggacttttaaagctggtggatgttgcagggcacgttgactctgggaccacactgcgctcatctcctgtttaa

SEQ ID NO: 18 - protein, 820 aa, Rat GST fusion
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDPMGHHHHHHGRRRASVAAGILVPRGSPGLDGICSIEEFRPPWKRANEGELKTGYLSIVMDPDELPLDERCERLPYDASKWEFPRDRLKLGKPLGRGAFGQVIEADAFGIDKTATCKTVAVKMLKEGATHSEHRALMSELKILIHIGHHLNVVNLLGACTKPGGPLMVIVEFCKFGNLSTYLRGKRNEFVPYKSKGARFRSGKDYVGELSVDLKRRLDSITSSQSSASSGFVEEKSLSDVEEEEASEELYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHEDPNQRPAFSELVEHLGNLLQANAQQDGKDYIVLPMSETLSMEEDSGLSLPTSPVSCMEEEEVCDPKFHYDNTAGISHYLQNSKRKSRPVSVKTFEDIPLEEPEVKVIPDDSQTDSGMVLASEELKTLEDRNKLSPSFGGMMPSKSRESVASEGSNQTSGYQSGYHSDDTDTTVYSSDEAGLLKLVDVAGHVDSGTTLRSSPV